(19) United States
(12) Patent Application Publication
(10) Pub. No.: US 2012/0141432 A1
(43) Pub. Date: Jun. 7, 2012

(54) USE OF CATALYTIC ANTIOXIDANT TO PRESERVE STEM CELL PHENOTYPE AND CONTROL CELL DIFFERENTIATION

(75) Inventors: Jon D. Piganelli, Pittsburgh, PA (US); Bridgette M. Deasy, Pittsburgh, PA (US); Steven M. Chirieleison, Pittsburgh, PA (US)

(73) Assignee: University of Pittsburgh- Of the Commonwealth System of Higher Education

(21) Appl. No.: 13/225,279

(22) Filed: Sep. 2, 2011

Related U.S. Application Data
(60) Provisional application No. 61/379,867, filed on Sep. 3, 2010.

(51) Int. Cl. A61K 35/12; C12N 5/09; C12N 5/0775; C12N 5/071; C12N 5/077

(52) U.S. Cl. 424/93.7; 435/377; 435/366

(57) ABSTRACT

Methods are disclosed herein for maintaining stem cells in an undifferentiated state in vitro. The methods include contacting the stem cells with an effective amount of a catalytic antioxidant. Also disclosed are methods for the increasing the number of stem cells in vitro while maintaining the stem cells in an undifferentiated state. The methods include contacting the stem cells with an effective amount of a catalytic antioxidant and an effective amount of one or more growth factors that promotes the expansion of the stem cells.

### CROSS REFERENCE TO RELATED APPLICATION

This application claims the benefit of U.S. Provisional Application No. 61/379,867, filed Sep. 3, 2010, which is incorporated herein in its entirety.

### ACKNOWLEDGMENT OF GOVERNMENT SUPPORT

This invention was made with government support under award number W18XWH-06-1-0406 awarded by the Department of Defense. The government has certain rights in the invention.

### FIELD

This disclosure relates to the field of catalytic antioxidants, and specifically to the use of catalytic antioxidants to maintain stem cells in an undifferentiated state.

### BACKGROUND

The use of stem cells and other progenitor cells in cell based therapeutics and regenerative medicine is a promising approach to the treatment of disease and injury. Although stem cells hold considerable promise for the treatment of a number of degenerative diseases, including Parkinson's disease and Duchenne Muscular Dystrophy, obstacles such as control of stem cell fate, and availability of large numbers of stem cells must be overcome before their therapeutic potential can be realized. One of the greatest challenges in area of stem cell based therapeutics is the control of stem cell fate during the production of large numbers of stem cells.

Stem cell fate is controlled by both intrinsic regulators and the extracellular environment, and is typically controlled in vitro by cell culture manipulation with “cocktails” of growth factors, signaling molecules, and/or by genetic manipulation. Propagation of stem cells on a large scale in a pure form under fully defined conditions and without accumulation of genetic damage in the cell is essential for production of sufficient numbers of cells for clinical utility. However, the expansion of stem cells in in vitro processing methods is often confounded by the spontaneous differentiation of stem cells. The intrinsic signals of the stem cells and progenitor cells often lead to differentiation even in defined media. Because cell-based therapies may require large quantities of stem cells for clinical use, it would be advantageous to utilize specific small molecules which can maintain self-renewal and in vitro expansion of the stem cells.

Therefore, the need exists for the development of small molecules that are capable of preventing stem cell differentiation. That need has now been met with the present disclosure.

### SUMMARY

Methods are disclosed herein for maintaining stem cells in an undifferentiated state in vitro. The methods include contacting the stem cells with an effective amount of a catalytic antioxidant, such as a porphyrin or a tetrapyrrole, or pharmaceutically acceptable salt thereof. In some non-limiting examples, the catalytic antioxidant is a porphyrin or a tetrapyrrole, such as FBC-007, or pharmaceutically acceptable salt thereof.

Also disclosed are methods for the increasing the number of stem cells in vitro while maintaining the stem cells in an undifferentiated state. The methods include contacting the stem cells with an effective amount of a catalytic antioxidant in an expansion media that promotes the expansion of the stem cell, such as an expansion media that includes an effective amount of one or more growth factors that promotes the expansion of the stem cells.

In some examples, the stem cells are transplanted into a subject, such as a subject that would benefit from a stem cell transplant.

Also disclosed are methods of testing the effect of an agent of interest on a stem cell, such as a cancer stem cell. In such methods, an expanded population of undifferentiated stem cells is contacted with an agent of interest and the effect of the agent of interest on expanded population of undifferentiated stem cells is determined.

The foregoing and features, and advantages of this disclosure will become more apparent from the following detailed description, which proceeds with reference to the accompanying figures.

### BRIEF DESCRIPTION OF THE DRAWINGS

FIG. 1 is the chemical structure of an exemplary catalytic antioxidant, FBC-007 also referred to as Mn(III)tetrakis(N-ethylpyridinium-2-yl)porphyrin (MnTE-2-PyP5+).

FIGS. 2A and 2B are a set of bar graphs showing the effect of the catalytic antioxidant FBC-007 on the differentiation of muscle stem cells. The effect of FBC-007 at a concentration of 34 μM was measured on the myogenic differentiation of muscle stem cells. Differentiation was determined as the % of nuclei located within myosin positive myotubes and multinucleated myotubes. FIG. 2A is a bar graph that shows that treatment with the catalytic antioxidant FBC-007 leads to reduced levels of differentiation relative to controls without the catalytic antioxidant FBC-007. FIG. 2B is a bar graph that shows that the division time of the muscle stem cells does not show a statistically significant change upon treatment with the catalytic antioxidant FBC-007. These results demonstrate that catalytic antioxidants are capable of maintaining stem cells in an undifferentiated state without the loss of phenotype while the stem cells expand.

FIG. 3 is a bar graph that shows the effect of the catalytic antioxidant FBC-007 on the osteogenic differentiation of human umbilical cord mesenchymal stem cells. The cells were contacted with the catalytic antioxidant at a concentration of 34 μM and bone morphogenic protein (BMP4) at concentrations of 0, 0.1, or 10 ng/mL. The stem cells showed reduced osteogenic differentiation in the presence of the catalytic antioxidant as compared to the control in which no antioxidant was present. This result demonstrates that a catalytic antioxidant, such as FCB-007, is capable of maintaining stem cells in an undifferentiated state.

FIG. 4 is a set of bar graphs showing that the muscle cell population size, N, was increased significantly (p<0.05) with epidermal growth factor (EGF), and also, to a lesser extent, by fibroblast growth factor 2 (FGF-2), Insulin-like growth factor 1 (IGF-1) (p<0.05) and stem cell factor (SCF).

FIGS. 5A-5D are bar graphs, a plot and an electronic image showing that oxidative stress decreases cell viability (FIG. 5A) and myogenic differentiation capacity (FIG. 5B). FIG. 5C shows that reactive oxygen species (ROS) increase in some muscle stem cells that are grown with FGF-2, but not in other cells which are grown in the absence of FGF-2. FIG. 5D is an electronic image of muscle cells showing that myosin (staining) declines as ROS increases.

FIGS. 6A-6C are a plot and a set of bar graphs showing that the catalytic antioxidant FBC-007 does not affect cell growth (FIGS. 6A and 6B), and prevents uninduced myogenic differentiation in the absence of growth factor (GF) stimulation (FIG. 6C).

FIGS. 7A-7E are plots and graphs that show that carboxyfluorescein diacetate succinimidyl ester (CFSE) identifies non-dividing cells and tracks cell division in dividing cells. FIG. 7A is a plot that shows cells stained with 5.0 μM CFSE that were subsequently UV-killed and added to a live unstained control population. The combined cells showed that no uptake of CFSE was observed. FIG. 7B is a graph that shows that CFSE does not affect growth rates. Cells treated with 0 μM, 1 μM, or 5 μM CFSE showed similar growth rates. FIG. 7C is a plot that shows that CFSE-stained muscle cells show a sequential loss of fluorescent intensity following initial staining. Cells were stained at 1.0 μM, and then analyzed at 0, 2, and 5 days. Note that the low fluorescence intensity approaches that of unstained cells by day 5. FIG. 7D is a plot of sample lineage trees showing the presence of dividing and non-dividing cells. FIG. 7E is a set of plots showing that 24 hours after injection into mdx mice, CFSE-labeled cells are detectable.

FIGS. 8A and 8B are electronic images showing the results of PCR and cell staining. FIG. 8A is an electronic image showing the results of PCR for myoD, myf5, myogenin, CD56 and desmin mRNA. FIG. 8B is an electronic image showing the results of cell staining. Staining shows heterogeneity in desmin and myoD cells. A short BrdU pulse reveals some dividing cells in the population. A decline in myogenic factors may occur with GF-expansion, ROS damage and cell aging.

FIGS. 9A-9C are electronic images of stained cells showing the progression of differentiation in human skMSCs as cells express desmin, myosin heavy chain and even dystrophin in vitro.

FIGS. 10A and 10B are a plot and a bar graph, respectively, showing muscle stem cell expansion potential. The greatest expansion potential is realized with skeletal muscle cell growth medium (SkGM™) (FIG. 10A), and not endothelial growth media-2 (EGM-2), which was previously shown to favor human myoendothelial cells. This demonstrates that optimization of cell expansion is cell-line specific and the human preplate cells can be expanded to high numbers using SkGM™ as compared to EGM-2 (FIG. 10A). Human skMSC also participate in muscle regeneration in mdx/SCID mice (FIG. 10B).

FIGS. 11A-11D show the effect of in vitro expansion on the quiescent and nondividing fraction and the regeneration of mouse MDSCs. FIG. 11A shows the stem cell expansion scheme. Mouse stem cell populations were expanded in the absence of GFs for >250 days, or ˜300 PDs. FIG. 11B shows that there is a decrease in the size of the nondividing fraction as cells are expanded in vitro. FIGS. 11C and 11D show that after extensive expansion, in vivo regeneration efficiency decreases.

FIGS. 12A-12F are chemical structures of specific prophyrin based catalytic antioxidants that can be used in the disclosed methods.

FIGS. 13A-13K are chemical structures of certain generic and specific definitions of compounds suitable for use in disclosed methods (in free or metal-bound forms). With reference to FIG. 13C, catalytic antioxidants of use in the disclosed methods can be of Formula I or II, or dimeric forms thereof, an example of which is shown in FIG. 13D.

### DETAILED DESCRIPTION

### I. Introduction

The use of stem cells and progenitor cells in cell based therapeutics and regenerative medicine is a promising approach to the treatment of disease and injury.

For example, muscle stem cell therapy is a promising approach for the treatment of skeletal muscle disorders such as Duchenne muscular dystrophy (DMD), sacropenia, sports injuries or trauma. However, for stem cell therapy to be effective, for example for muscle diseases, like DMD, it will likely be necessary to transplant large numbers of cells in order to reach more than 600 muscles or to replace muscle mass in the case of tissue loss due to traumatic injury. Challenges in obtaining clinically relevant cell doses arise from both the insufficient number of stem cells harvested from the source, for example an stem cell donor, and also limited in vitro proliferative capacity of human stem cells, for example to increase the numbers of stem cell for transplantation. Unfortunately, current methods of stem cell expansion induce age-related changes in stem cell populations, such as a differentiation and loss of the phenotypic characteristics that distinguish stem cells from other cells. Thus, the need exists for new methods of in vitro expansion of stem cells, while maintaining the expanded stem cells in an undifferentiated state, such that the phenotypic characteristics that distinguish stem cells from other cells are not lost during expansion.

As disclosed herein, age-related changes that affect the ability of stem cells to maintain an undifferentiated state and the phenotypic characteristics that distinguish stem cells from other cells are due in part to both an increase in reactive oxygen species (ROS) within the cells.

The use of a catalytic antioxidant, for example FB-007, inhibits the myogenic differentiation in stem cell cultures without affecting the proliferation of the stem cells, such as muscle stem cell and mesenchymal stem cells. In other words, contacting stem cells with catalytic antioxidants allows the stem cells to proliferate (e.g. increase their numbers) without differentiating and losing the characteristics of stem cells, such as the phenotypic characteristics that distinguish stem cells from other cells. In addition, the use of catalytic antioxidants, such as FBC-007, can rescue the stem cells from hydrogen peroxide-induce cell death. Thus, as disclosed herein contacting stem cells with catalytic antioxidants permits in vitro expansion of stem cells while delaying or inhibiting the differentiation of the stem cells, as well as protecting the stem cells from oxidative stress induced cell death.

### II. Summary of Terms

Unless otherwise noted, technical terms are used according to conventional usage. Definitions of common terms in molecular biology may be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710).

The singular terms “a,” “an,” and “the” include plural referents unless context clearly indicates otherwise. For example, the term “a stem cell” includes single or plural stem cells and can be considered equivalent to the phrase “at least one stem cell.” Similarly, the word “or” is intended to include “and” unless the context clearly indicates otherwise. The term “comprises” means “includes.” For example, “comprising a stem cell” means “including a stem cell” without excluding other elements.

In case of conflict, the present specification, including explanations of terms, will control. To facilitate review of the various embodiments of this disclosure, the following explanations of terms are provided.

Administration: The introduction of a composition into a subject by a chosen route. For example, if the chosen route is intravenous, the composition (such as a stem cell) is administered by introducing the composition into a vein of the subject. In some examples, administration of stem cells includes the transplantation of stem cells into a subject, such as a human subject.

Animal: A living multi-cellular vertebrate or invertebrate organism, a category that includes, for example, mammals. The term mammal includes both human and non-human mammals. Similarly, the term “subject” includes both human and veterinary subjects, such as non-human primates. Thus, administration to a subject can include administration to a human subject. Particular examples of veterinary subjects include domesticated animals (such as cats and dogs), livestock (for example, cattle, horses, pigs, sheep, and goats), and laboratory animals (for example, mice, rabbits, rats, gerbils, guinea pigs, and non-human primates).

Cancer: A malignant disease characterized by the abnormal growth and differentiation of cells. “Metastatic disease” refers to cancer cells that have left the original tumor site and migrate to other parts of the body for example via the bloodstream or lymph system. In some examples, a cancer stem cell is obtained from a cancer, such as from a hematological tumor or a solid tumor.

Examples of hematological tumors include leukemias, for example acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia, and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (such as adenocarcinoma), lung cancers, gynecological cancers (such as, cancers of the uterus (e.g., endometrial carcinoma), cervix (e.g., cervical carcinoma, pre-tumor cervical dysplasia), ovaries (e.g., ovarian carcinoma, serous cystadenocarcinoma, mucinous cystadenocarcinoma, endometrioid tumors, celioblastoma, clear cell carcinoma, unclassified carcinoma, granulosa-thecal cell tumors, Sertoli-Leydig cell tumors, dysgerminoma, malignant teratoma), vulva (e.g., squamous cell carcinoma, intraepithelial carcinoma, adenocarcinoma, fibrosarcoma, melanoma), vagina (e.g., clear cell carcinoma, squamous cell carcinoma, botryoid sarcoma), embryonal rhabdomyosarcoma, and fallopian tubes (e.g., carcinoma)), prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma), and skin cancer (such as melanoma and non-melonoma). In some examples a cancer cell is a cancer stem cell, such as a cancer stem cell isolated from a subject.

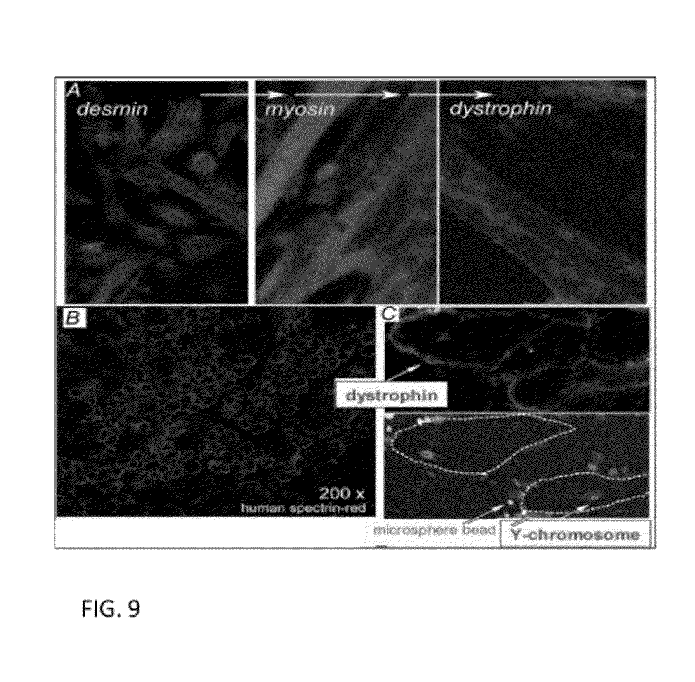

Catalytic Antioxidant: Synthetic molecules intended to catalyze the reduction or decomposition of reactive oxygen species (ROS), thereby providing protection against oxidative stress. This category of antioxidants includes metal porphyrins and mimics of superoxide dismutase and catalase activities. In some examples, catalytic antioxidants are metalloporphyrins, see U.S. Patent Publication No. 2003/0032634, which is incorporated herein by reference in its entirety. Specific examples of catalytic antioxidants can be found in FIGS. 9 and 18 of U.S. Patent Publication No. 2003/0032634.

Cell cycle: The physiological and morphological progression of changes that cells undergo when dividing. The cell cycle consists of a cell division phase and the events that occur during the period between successive cell divisions, known as interphase. Interphase is composed of successive G1, S, and G2 phases, and normally comprises 90% or more of the total cell cycle time. Most cell components are made continuously throughout interphase. It is therefore difficult to define distinct stages in the progression of the growing cell through interphase. One exception is DNA synthesis, since the DNA in the cell nucleus is replicated only during a limited portion of interphase. This period is denoted as the S phase (S=synthesis) of the cell cycle. The other distinct stage of the cell cycle is the cell division phase, which includes both nuclear division (mitosis) and the cytoplasmic division (cytokinesis) that follows. The entire cell division phase is denoted as the M phase (M=mitotic). This leaves the period between the M phase and the start of DNA synthesis, which is called the G1 phase (G=gap), and the period between the completion of DNA synthesis and the next M phase, which is called the G2 phase (Alberts et al., Molecular Biology of the Cell, New York: Garland Publishing, Inc., 1983, pages 611-612).

Chemotherapeutic agents: Any chemical agent with therapeutic usefulness in the treatment of diseases characterized by abnormal cell growth. Such diseases include tumors, neoplasms, and cancer as well as diseases characterized by hyperplastic growth such as psoriasis. Chemotherapeutic agents are described for example in Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993. Combination chemotherapy is the administration of more than one agent to treat cancer.

Collecting: Refers to the process of removing stem cells from a subject, such as a human subject. Collecting optionally includes separating the stem cells from other cell types. Collection and separation of stem cells is well known in the art. Separation can be done based on the expression of phenotypic markers, such as cell surface markers that distinguish stem cells from non-stem cells, for example using flow cytometry or magnetic bead purification among others.

Differentiation: The process by which cells become more specialized to perform biological functions. As stem cells undergo the process of differentiation, they lose the properties of stem cells. For example once a cell has committed to a specific lineage (such as becoming a muscle cell), the cell is no longer a stem cell. However, the differentiated cell may still be a progenitor cell for other cell types within the lineage tree.

Effective amount: An amount sufficient to evoke a desired response from a cell of interest. In one embodiment, an effective amount of an agent is the amount sufficient to affect the proliferation or differentiation of a cell, such as a stem cell, for example an effective amount of a catalytic antioxidant to inhibit differentiation of a stem cell.

Embryonic stem (ES) cells: Pluripotent cells isolated from the inner cell mass of the developing blastocyst. ES cells are pluripotent cells, meaning that they can generate all of the cells present in the body (bone, muscle, brain cells, etc.). Methods for producing murine ES cells can be found in U.S. Pat. No. 5,670,37. Methods for producing human ES cells can be found in U.S. Pat. No. 6,090,622, PCT Publication No. WO 00/70021 and PCT Publication No. WO 00/27995.

Epidermal growth factor (EGF): A globular protein of 6.4 kDa including 53 amino acids. It contains three intramolecular disulfide bonds. EGF proteins are evolutionarily closely conserved. Human EGF and murine EGF have 37 amino acids in common. Approximately 70 percent homology is found between human EGF and EGF isolated from other species. Mammalian EGF includes, but is not limited to, murine, avian, canine, bovine, porcine, equine, and human EGF. The amino acid sequences and methods for making these EGF polypeptides are well known in the art.

The gene encoding the EGF precursor has a length of approximately 110 kb, and contains 24 exons. Fifteen of these exons encode protein domains that are homologous to domains found in other proteins. The human EGF gene maps to chromosome 4q25-q27.

EGF is a strong mitogen for many cells of ectodermal, mesodermal, and endodermal origin. EGF controls and stimulates the proliferation of epidermal and epithelial cells, including fibroblasts, kidney epithelial cells, human glial cells, ovary granulosa cells, and thyroid cells in vitro. EGF also stimulates the proliferation of embryonic cells. However, the proliferation of some cell lines has been shown to be inhibited by EGF.

EGF is also known to act as a differentiation factor for some cell types. It strongly influences the synthesis and turn-over of proteins of the extra-cellular matrix including fibronectin, collagen, laminin, and glycosaminoglycans, and has been shown to be a strong chemoattractant for fibroblasts and epithelial cells.

Fragments of EGF, smaller than the full-length sequence can also be employed in methods disclosed herein. Suitable biologically active variants can also be utilized. One specific, non-limiting example of an EGF variant of use is an EGF sequence having one or more amino acid substitutions, insertions, or deletions, wherein a biological function of EGF is retained. Another specific, non-limiting example of an EGF variant is EGF as wherein glycosylation or phosphorylation is altered, or a foreign moiety is added, so long as a biological function of EGF is retained. Methods for making EGF fragments, analogues, and derivatives are available in the art. Examples of EGF variants are known in the art, for example U.S. Pat. No. 5,218,093 and WO 92/16626A1. Examples of EGF from many different species are disclosed in WO 92/16626A1, as are examples of variants, and strategies for producing them.

As used herein, “EGF” refers to naturally occurring EGF, and variants and fragments that perform the same function of EGF in the culture media disclosed herein.

Expand: To increase in quantity. As used herein, “expanding” stem cells, such as human stem cells, for example human muscle stem cells or human umbilical cord mesenchymal stem cells, refers to the process of allowing cell division to occur such that the number of the stem cells increases. Using the methods described herein, it is possible to expand stem cells in culture without the cells losing the characteristics of stem cells, such as the characteristics that phenotypically distinguish stem cells from other cells. The terms “proliferate,” “proliferation” or “proliferated” may be used interchangeably with the words “expand,” “expansion”, or “expanded.” During an expansion phase, the stem cells do not differentiate to form mature cells, but divide to form more, non-differentiated stem cells.

Fibroblast growth factor or FGF: Any suitable fibroblast growth factor, derived from any animal, and functional fragments thereof. A variety of FGFs are known and include, but are not limited to, FGF-1 (acidic fibroblast growth factor), FGF-2 (basic fibroblast growth factor, bFGF), FGF-3 (int-2), FGF-4 (hst/K-FGF), FGF-5, FGF-6, FGF-7, FGF-8, FGF-9 and FGF-98. “FGF” refers to a fibroblast growth factor protein such as FGF-1, FGF-2, FGF-4, FGF-6, FGF-8, FGF-9 or FGF-98, or a biologically active fragment or mutant thereof. The FGF can be from any animal species. In one embodiment, the FGF is mammalian FGF, including but not limited to, rodent, avian, canine, bovine, porcine, equine and human. The amino acid sequences and method for making many of the FGFs are well known in the art.

The amino acid sequence of human FGF-1 and a method for its recombinant expression are disclosed in U.S. Pat. No. 5,604,293. The amino acid sequence of human FGF-2 and methods for its recombinant expression are disclosed in U.S. Pat. No. 5,439,818. The amino acid sequence of bovine FGF-2 and various methods for its recombinant expression are disclosed in U.S. Pat. No. 5,155,21. When the 146 residue forms are compared, their amino acid sequences are nearly identical, with only two residues that differ.

The amino acid sequence of FGF-3 (Dickson et al., Nature 326:833, 1987) and human FGF-4 (Yoshida et al., PHAS USA 84:7305-7309, 1987) are known. When the amino acid sequences of human FGF-4, FGF-1, FGF-2 and murine FGF-3 are compared, residues 72-204 of human FGF-4 have 43% homology to human FGF-2; residues 79-204 have 38% homology to human FGF-1; and residues 72-174 have 40% homology to murine FGF-3. The cDNA and deduced amino acid sequences for human FGF-5 (Zhan et al., Molec. and Cell. Biol. 8(8):3487-3495, 1988), human FGF-6 (Coulier et al., Oncogene 6:1437-1444, 1991), human FGF-7 (Miyamoto et al., Mol. and Cell. Biol. 13(7):4251-4259, 1993) are also known. The cDNA and deduced amino acid sequence of murine FGRF-8 (Tanaka et al., PNAS USA 89:8928-8932, 1992), human and murine FGF-9 (Santos-Ocamp et al., J. Biol. Chem. 271(3):1726-1731, 1996) and human FGF-98 (provisional patent application Ser. No. 60/083,553, which is hereby incorporated herein by reference in its entirety) are also known.

FGF-2 (also known as bFGF or bFGF-2), and other FGFs, can be made as described in U.S. Pat. No. 5,155,214 (“the '214 patent”). The recombinant bFGF-2, and other FGFs, can be purified to pharmaceutical quality (98% or greater purity) using the techniques described in detail in U.S. Pat. No. 4,956,455.

Growth factor: A substance that promotes cell growth, survival, and/or differentiation. Growth factors include molecules that function as growth stimulators (mitogens), molecules that function as growth inhibitors (e.g. negative growth factors) factors that stimulate cell migration, factors that function as chemotactic agents or inhibit cell migration or invasion of tumor cells, factors that modulate differentiated functions of cells, factors involved in apoptosis, or factors that promote survival of cells without influencing growth and differentiation. Examples of growth factors are a fibroblast growth factor (such as FGF-2), epidermal growth factor (EGF), cilliary neurotrophic factor (CNTF), and nerve growth factor (NGF), insulin-like growth factor 1 (IGF-1), FLT-3 ligand, stem cell factor (SCF) and actvin-A.

Genome stability: The ability of a cell to faithfully replicate DNA and maintain integrity of the DNA replication machinery. A stem cell (such as a human stem cell, for example a human muscle stem cell or human umbilical cord mesenchymal stem cell) with a stable genome generally defies cellular senescence, can proliferate more than 10 doublings, such as at least 50 doublings, at least 100 doublings, at least 150 doublings, at least 200 doublings, at least 250 doublings, at least 300 doublings, at least 400 doublings, at least 500 doublings or at least 1000 doublings or even greater than 1000 doublings without undergoing crisis or transformation, has a low mutation frequency and a low frequency of chromosomal abnormalities, and maintains genomic integrity. Long telomeres are thought to provide a buffer against cellular senescence and be generally indicative of genome stability and overall cell health. Chromosome stability, which includes the introduction of few mutations, no chromosomal rearrangements, or changes in chromosomal number, is also associated with genome stability. A loss of genome stability can be associated with cellular aging, for example the aging of a cell population as it goes through successive doublings. Signs of genome instability include elevated mutation rates, gross chromosomal rearrangements, alterations in chromosome number, and shortening of telomeres.

Growth medium or expansion medium: A synthetic set of culture conditions with the nutrients necessary to support the growth (for exmaple cell proliferation/expansion) of a specific population of cells such as stem cells. In one embodiment, the cells are stem cells, such as human muscle stem cells or human umbilical cord mesenchymal stem cells. Growth media generally include a carbon source, a nitrogen source and a buffer to maintain pH. In one embodiment, growth medium contains a minimal essential media, such as DMEM, supplemented with various nutrients to enhance stem cell growth. Additionally, the minimal essential media may be supplemented with additives such as horse, calf or fetal bovine serum and growth factors, such as EGF, FGF-2, IGF-1, FLT-3 ligand, or SCF. In some examples, growth or expasion medium contians an efective amount of a catalytic antioxidant.

Induced pluripotent stem (iPS) cells: A type of pluripotent stem cell artificially derived from a non-pluripotent cell, typically an adult somatic cell, by inducing a “forced” expression of certain genes. iPS cells can be derived from any organism, such as a mammal. In one embodiment, iPS cells are produced from mice, rats, rabbits, guinea pigs, goats, pigs, cows, monkeys and humans.

iPS cells are similar to ES cells in many respects, such as the expression of certain stem cell genes and proteins, chromatin methylation patterns, doubling time, embryoid body formation, teratoma formation, viable chimera formation, and potency and differentiability. Methods for producing iPS cells are known in the art. For example, iPS cells are typically derived by transfection of certain stem cell-associated genes (such as Oct-3/4 (Pouf51) and Sox2) into non-pluripotent cells, such as adult fibroblasts. Transfection can be achieved through viral vectors, such as retroviruses, lentiviruses, or adenoviruses. For example, cells can be transfected with Oct3/4, Sox2, Klf4, and c-Myc using a retroviral system or with OCT4, SOX2, NANOG, and LIN28 using a lentiviral system. In one example, iPS from adult human cells are generated by the method of Yu et al., (Science 318(5854):1224, 2007) or Takahashi et al., (Cell 131(5):861-72, 2007).

Inhibit: A decrease in a particular parameter of a cell or organism, for example inhibiting differentiation of a stem cell.

In vitro: Occurring outside of a living organism. For example a procedure performed in vitro (such as the in vitro expansion of stem cells) is performed not in a living organism but in a controlled environment, such as in tissue culture.

Isolated: An “isolated” biological component (such as a stem cell, for example a human stem cell, for example human muscle or human umbilical cord mesenchymal stem cell, has been substantially separated or purified away from other biological components in which the component naturally occurs, such as other cells and extra cellular milieu. Isolated does not require absolute purity, and can include cell preparations, such as stem cell preparations, that are at least 50% isolated, such as at least 75%, 80%, 90%, 95%, 98%, 99%, or even 100% isolated.

Mesenchymal Stem Cell: A cell that proliferates in vitro that is undifferentiated and, under appropriate culture conditions, can differentiate into cells of a defined lineage.

Muscle cell: A cell of striated, cardiac, or smooth muscle tissue. In striated (skeletal) muscle, a muscle cell is composed of a syncytium formed by the fusion of embryonic myoblasts. In smooth muscle, a muscle cell is a single cell characterized by large amounts of actin and myosin and capable of contracting to a small fraction of its overall length. In cardiac muscle, the muscle cell is linked to neighboring cells by specialized junctions called intercalated discs.

Pharmaceutically acceptable salt: Salts formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002).

Pluripotent cell: A cell with the potential to differentiate into cells of the three germ layers: endoderm (for example interior stomach lining, gastrointestinal tract, and the lungs), mesoderm (for example muscle, bone, blood, and urogenital), or ectoderm (for example epidermal tissues and nervous system). Pluripotent stem cells can give rise to any fetal or adult cell type. Alone they cannot develop into a fetal or adult animal because they lack the potential to contribute to extraembryonic tissue (for example placenta in vivo or trophoblast in vitro).

Pluripotent stem cells (PSCs) are the source of multipotent stem cells (MPSCs) through spontaneous differentiation or as a result of exposure to differentiation induction conditions in vitro. The term “multipotent” refers to a cell's potential to differentiate and give rise to a limited number of related, different cell types. These cells are characterized by their multi-lineage potential and the ability for self-renewal. In vivo, the pool of multipotent stem cells replenishes the population of mature functionally active cells in the body. Among the exemplary multipotent stem cell types are hematopoietic, mesenchymal, or neuronal stem cells.

Transplantable cells include MPSCs and more specialized cell types such as committed progenitors as well as cells further along the differentiation and/or maturation pathway that are partly or fully matured or differentiated. “Committed progenitors” give rise to a fully differentiated cell of a specific cell lineage. Exemplary transplantable cells include pancreatic cells, epithelial cells, cardiac cells, endothelial cells, liver cells, endocrine cells, and the like.

Precursor Cell: A cell that can generate a fully differentiated functional cell of at least one given cell type. Generally, precursor cells can divide. After division, a precursor cell can remain a precursor cell, or may proceed to terminal differentiation. A “muscle precursor cell” is a precursor cell that can generate a fully differentiated functional muscle cell, such as a cardiomyocyte or a skeletal muscle cell. One specific, non-limiting example of a muscle precursor cell is a “cardiac precursor cell,” which is a cell that gives rise to cardiac muscle cells.

Progenitor cell: A cell that gives rise to progeny in a defined cell lineage.

Prolonging viability: As used herein, “prolonging viability” of a stem cell refers to extending the duration of time a stem cell is capable of normal growth and/or survival. In some examples, the disclosed methods are used to prolong the viability of stem cells in an undifferentiated state.

Purified: The term purified does not require absolute purity; rather, it is intended as a relative term. Thus, for example, a purified stem cell preparation is one in which the preparation is more enriched in stem cells than is in its natural environment within a subject. Preferably, a preparation is purified such that the stem cells represent at least 50% of the cellular content of the preparation.

Reactive Oxygen Species (ROS): Reactive oxygen species (ROS) are cytotoxic and mutagenic. ROS modify and damage critical biomolecules including DNA, protein, and lipids. They are partial reduction products of oxygen: 1 electron reduces O₂ to form superoxide (O₂⁻), and 2 electrons reduce O₂ to form hydrogen peroxide (H₂O₂). The cytotoxic property of ROS is exploited by phagocytes, which generate large amounts of superoxide and hydrogen peroxide as part of their armory of bactericidal mechanisms. ROS have been considered an accidental byproduct of metabolism, particularly mitochondrial respiration. Recent studies give evidence for regulated enzymatic generation of O₂⁻, and its conversion to H₂O₂ in a variety of cells.

Several biological systems generate reactive oxygen. For example, exposure of neutrophils to bacteria or to various soluble mediators such as formyl-Met-Leu-Phe or phorbol esters activates a massive consumption of oxygen, termed the respiratory burst, to initially generate superoxide, with secondary generation of H₂O₂, HOCl and hydroxyl radical. The enzyme responsible for this oxygen consumption is the respiratory burst oxidase (nicotinamide adenine dinucleotide phosphate-reduced form (NADPH) oxidase).

Skeletal muscle: Skeletal muscle makes up most of the body's muscle and does not contract without nervous stimulation. It is under voluntary control and lacks anatomic cellular connections between fibers. The fibers (cells) are multinucleate and appear striated due to the arrangement of actin and myosin protein filaments. Each fiber is a single cell, long, cylindrical and surrounded by a cell membrane. The muscle fibers contain many myofibrils that are made of myofilaments. These myofilaments are made up of contractile proteins. The key proteins in muscle contraction are myosin, actin, tropomyosin and troponin.

Stem cell: A cell having the unique capacity to produce unaltered daughter cells (self-renewal; cell division produces at least one daughter cell that is identical to the parent cell) and to give rise to specialized cell types (potency). Stem cells include, but are not limited to, embryonic stem (ES) cells, embryonic germ (EG) cells, germline stem (GS) cells, human mesenchymal stem cells (hMSCs), adipose tissue-derived stem cells (ADSCs), multipotent adult progenitor cells (MAPCs), multipotent adult germline stem cells (maGSCs) and unrestricted somatic stem cell (USSCs). The role of stem cells in vivo is to replace cells that are destroyed during the normal life of an animal. Generally, stem cells can divide without limit. After division, the stem cell may remain as a stem cell, become a precursor cell, or proceed to terminal differentiation. A precursor cell is a cell that can generate a fully differentiated functional cell of at least one given cell type. Generally, precursor cells can divide. After division, a precursor cell can remain a precursor cell, or may proceed to terminal differentiation.

When a stem cell divides, each new cell has the potential either to remain a stem cell or become another type of cell with a more specialized function, such as a muscle cell, a red blood cell, or a brain cell.

Stem cells are distinguished from other cell types by two important characteristics. First, they are unspecialized cells capable of renewing themselves through cell division, sometimes after long periods of inactivity. Second, under certain physiologic or experimental conditions, they can be induced to become tissue- or organ-specific cells with special functions. In some organs, such as the gut and bone marrow, stem cells regularly divide to repair and replace worn out or damaged tissues. In other organs, however, such as the pancreas and the heart, stem cells only divide under special conditions. When unspecialized stem cells give rise to specialized cells, the process is called differentiation. While differentiating, the cell usually goes through several stages, becoming more specialized at each step. The external signals for cell differentiation include chemicals secreted by other cells, physical contact with neighboring cells, and certain molecules in the microenvironment. The interaction of signals during differentiation causes the cell's DNA to acquire epigenetic marks that restrict DNA expression in the cell and can be passed on through cell division.

An adult stem cell is thought to be an undifferentiated cell, found among differentiated cells in a tissue or organ that can renew itself and can differentiate to yield some or all of the major specialized cell types of the tissue or organ. The primary roles of adult stem cells in a living organism are to maintain and repair the tissue in which they are found. The term somatic stem cell can be used instead of adult stem cell, where somatic refers to cells of the body (not the germ cells, sperm or eggs).

Hematopoietic stem cells give rise to all the types of blood cells: red blood cells, B lymphocytes, T lymphocytes, natural killer cells, neutrophils, basophils, eosinophils, monocytes, and macrophages.

Mesenchymal stem cells give rise to a variety of cell types: bone cells (osteocytes), cartilage cells (chondrocytes), fat cells (adipocytes), and other kinds of connective tissue cells such as those in tendons.

Neural stem cells in the brain give rise to its three major cell types: nerve cells (neurons) and two categories of non-neuronal cells—astrocytes and oligodendrocytes.

Epithelial stem cells in the lining of the digestive tract occur in deep crypts and give rise to several cell types: absorptive cells, goblet cells, paneth cells, and enteroendocrine cells.

Skin stem cells occur in the basal layer of the epidermis and at the base of hair follicles. The epidermal stem cells give rise to keratinocytes, which migrate to the surface of the skin and form a protective layer. The follicular stem cells can give rise to both the hair follicle and to the epidermis.

Senescence: The inability of a cell to divide further. A senescent cell is still viable, but does not divide.

Subpopulation: An identifiable portion of a population, for example, a subpopulation of cells, stem cells, or human stem cells.

Suspension: A dispersion of solid particles, such as a cell, throughout the body of a liquid, such as a culture medium or an isotonic (physiologically compatible) buffer.

Totipotent cell: Refers to a cell that can form an entire organism autonomously. The term “totipotent” or “totipotency” refers to a cell's ability to divide and ultimately produce an organism and its extraembryonic tissues in vivo. In one aspect, the term “totipotent” refers to the ability of the cell to progress through a series of divisions into a blastocyst in vitro. The blastocyst includes an inner cellular mass (ICM) and a trophoblast. By ICM it is meant the cells surrounded by the trophectoderm. The inner cell mass cells give rise to most of the fetal tissues upon further development. The cells found in the ICM give rise to pluripotent stem cells that possess the ability to proliferate indefinitely, or if properly induced, differentiate in all cell types contributing to an organism. “Trophectoderm” is the outermost layer of cells surrounding the blastocoel during the blastocyst stage of primate embryonic development. Trophectoderm becomes trophoblast and gives rise to most or all of the placental tissue upon further development. Trophoblast cells generate extra-embryonic tissues, including placenta and amnion. TSCs are the source of PSCs.

Telomere: The sequences and the ends of a eukaryotic chromosome, consisting of many repeats of a short DNA sequence in specific orientation. Telomere functions include protecting the ends of the chromosome, so that chromosomes do not end up joined together, and allowing replication of the extreme ends of the chromosomes (by telomerase). The number of repeats of telomeric DNA at the end of a chromosome decreases with age and telomeres plays a role in cellular aging roles in aging.

Telomerase: A DNA polymerase involved in the formation of telomeres and the maintenance of telomere sequences during chromosome replication.

Suitable methods and materials for the practice or testing of this disclosure are described below. Such methods and materials are illustrative only and are not intended to be limiting. Other methods and materials similar or equivalent to those described herein can be used. For example, conventional methods well known in the art to which a disclosed invention pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999. In addition, the materials, methods, and examples are illustrative only and not intended to be limiting.

### III. Overview of Several Embodiments

A. Catalytic Antioxidants

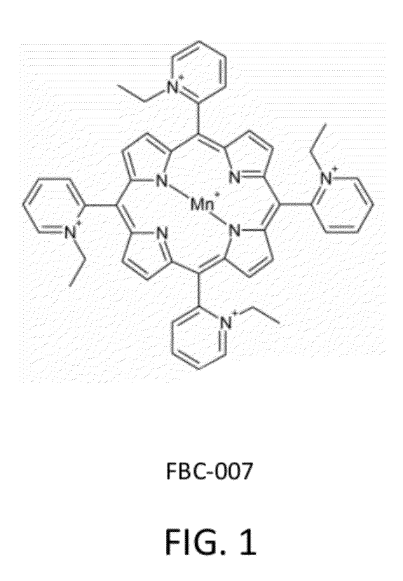

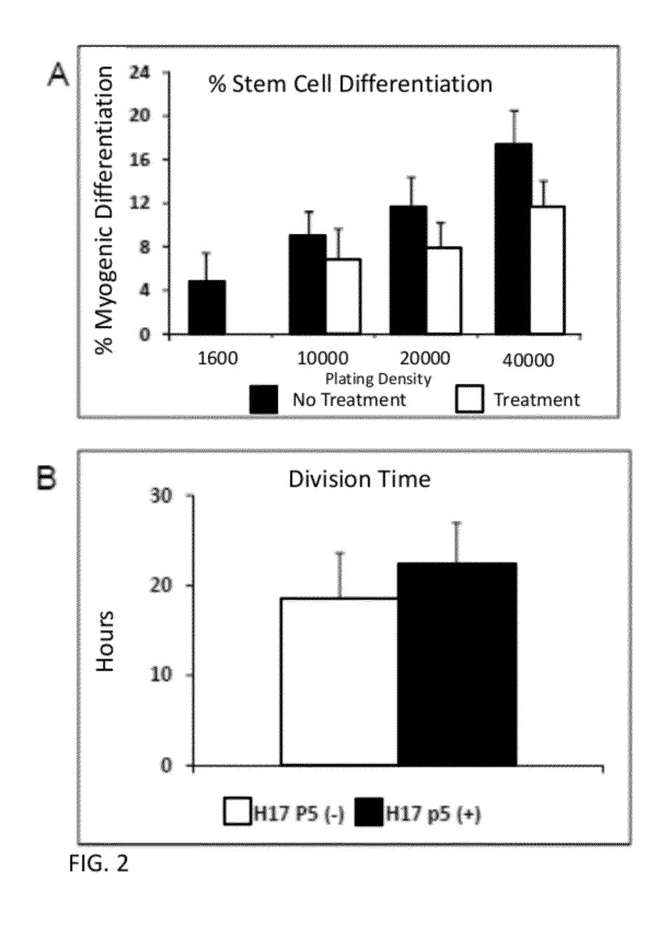

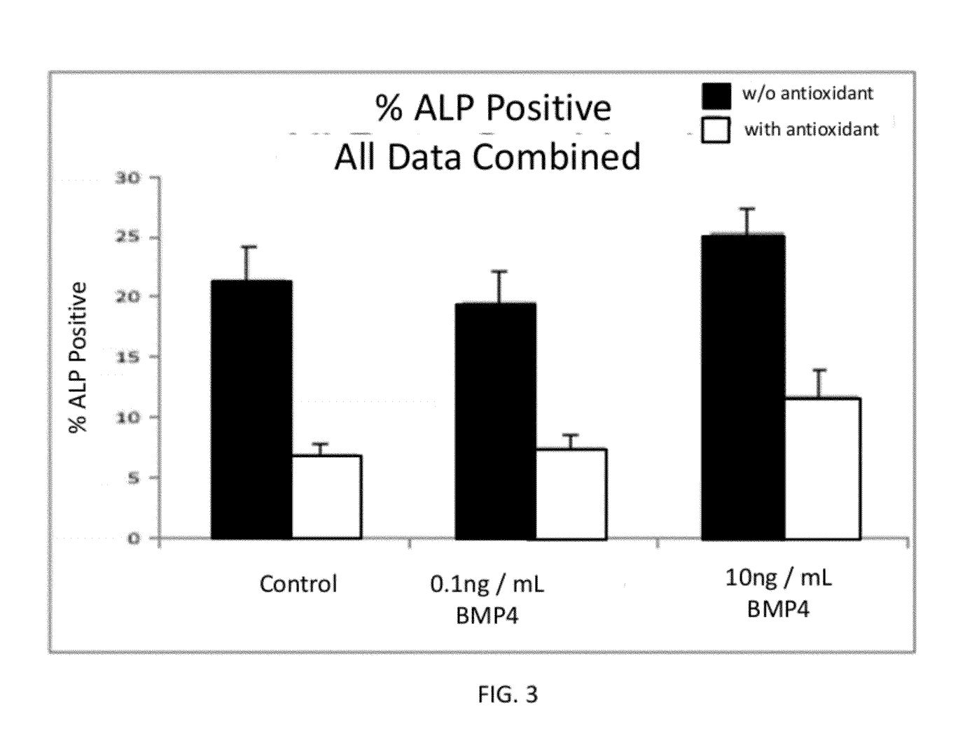

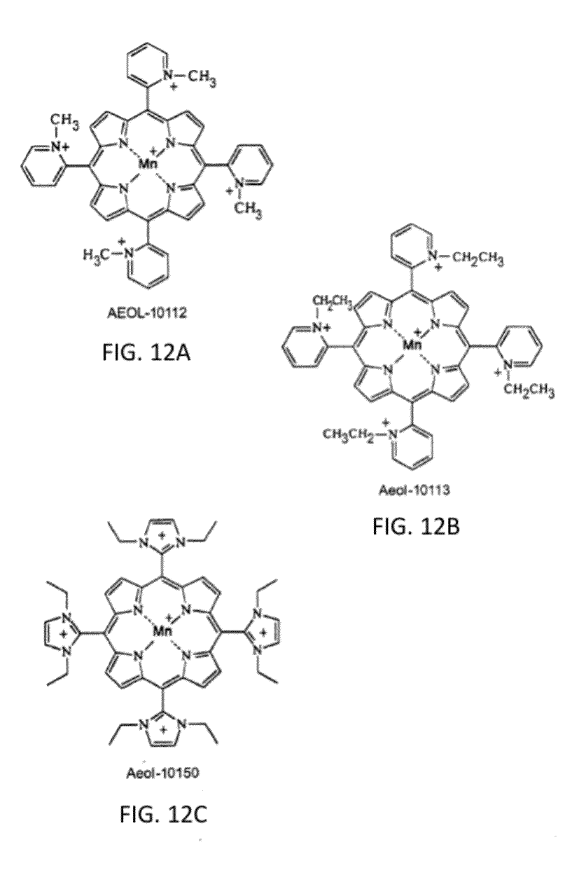

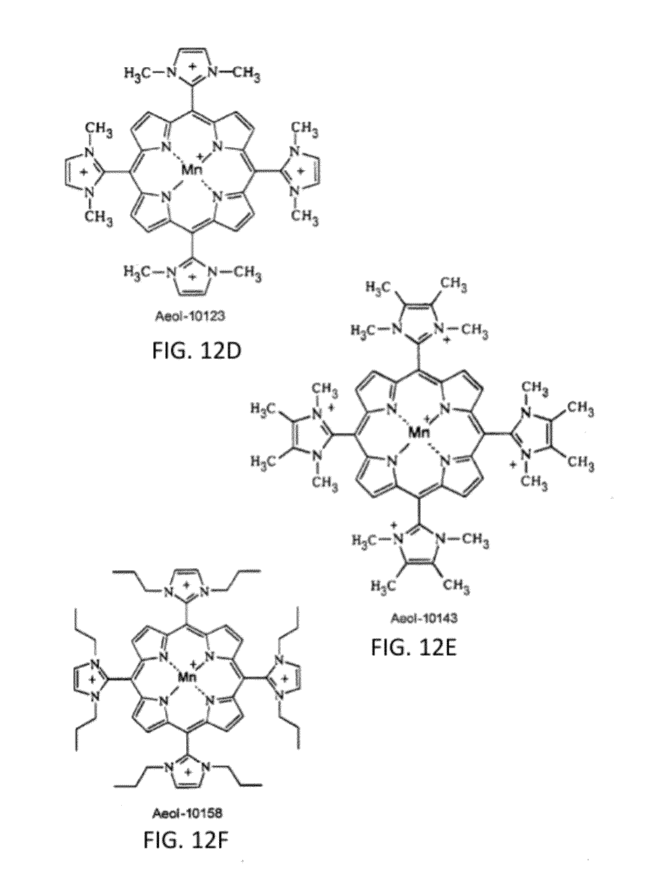

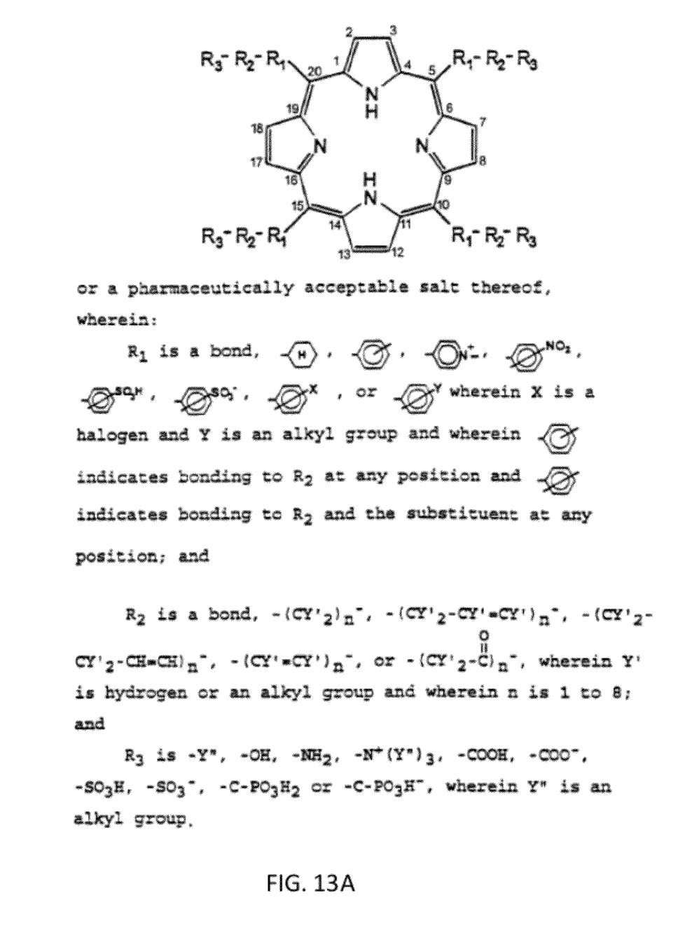

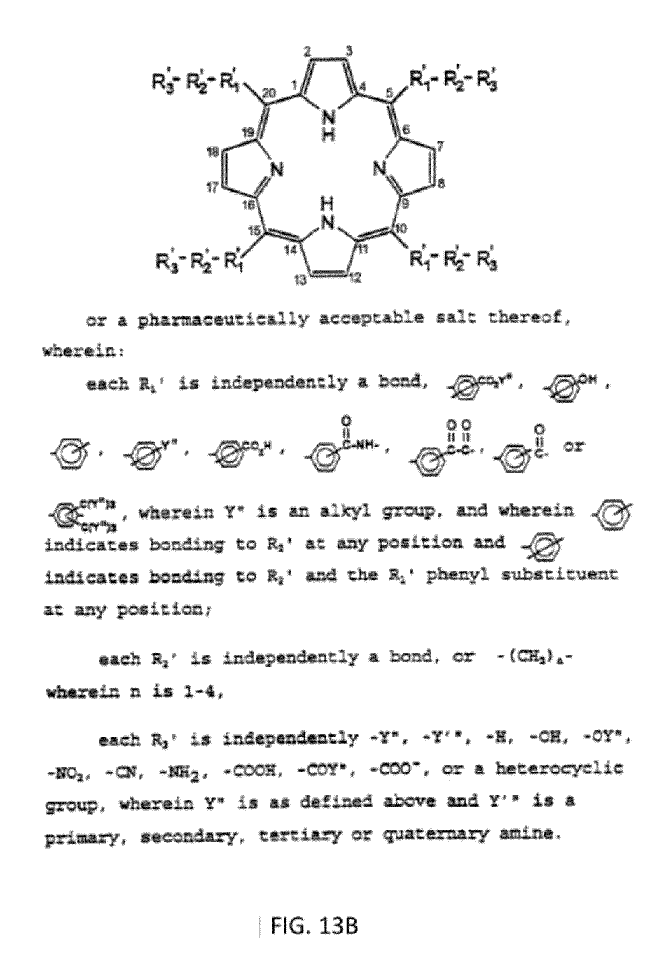

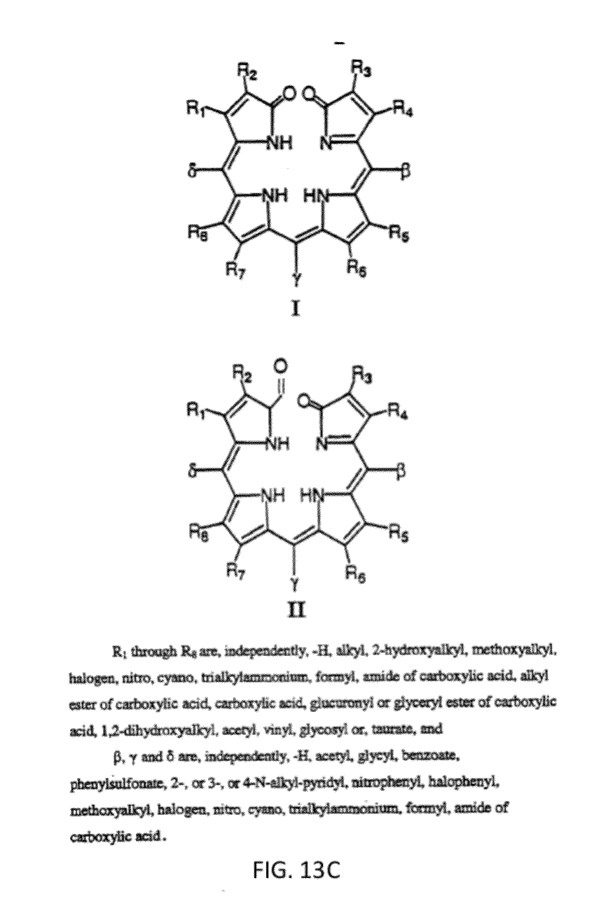

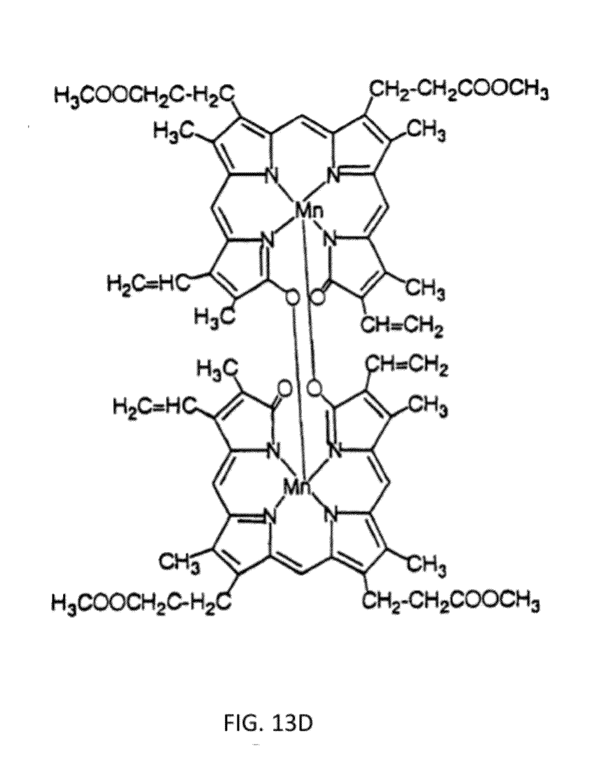

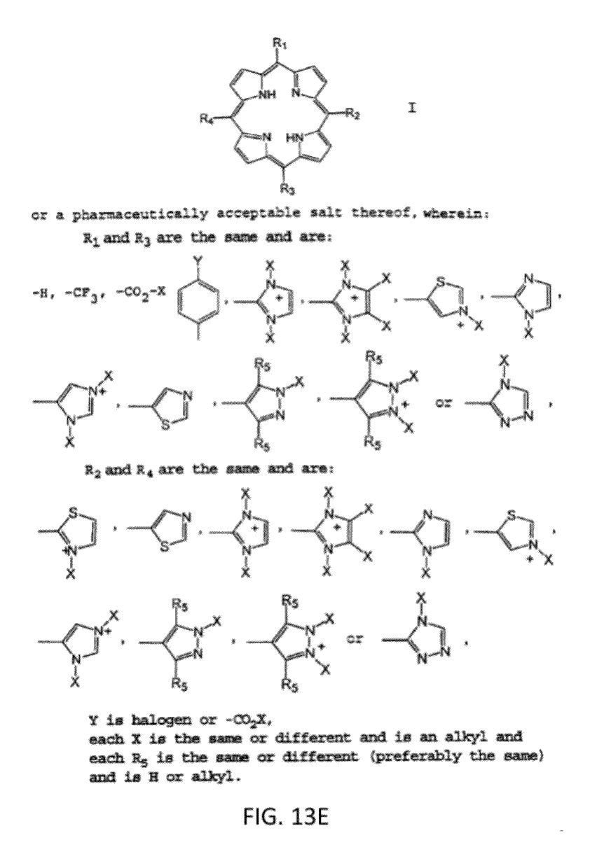

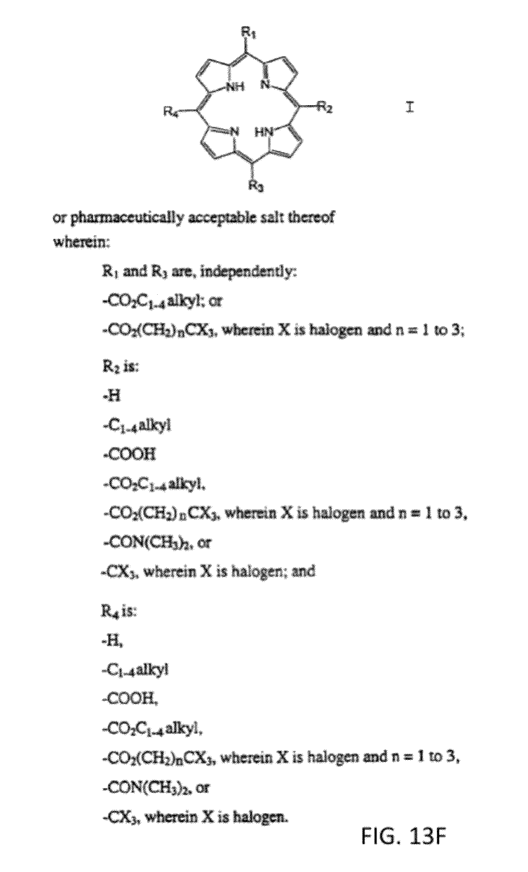

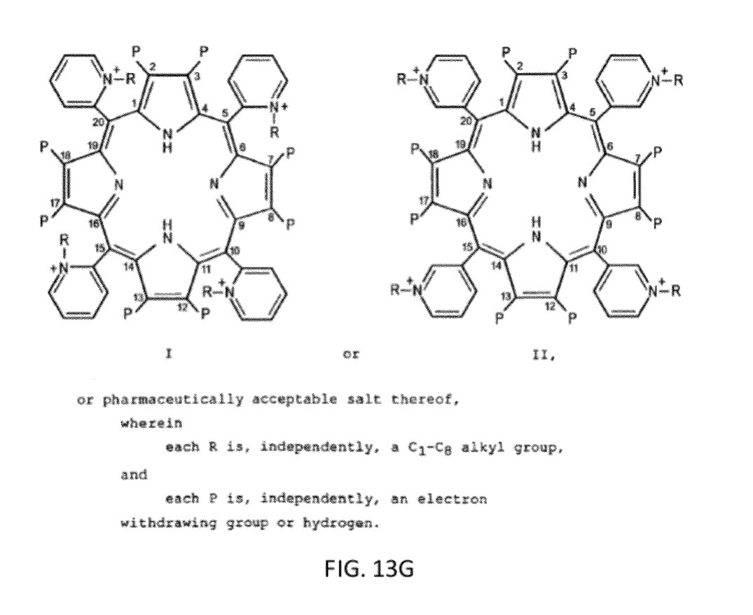

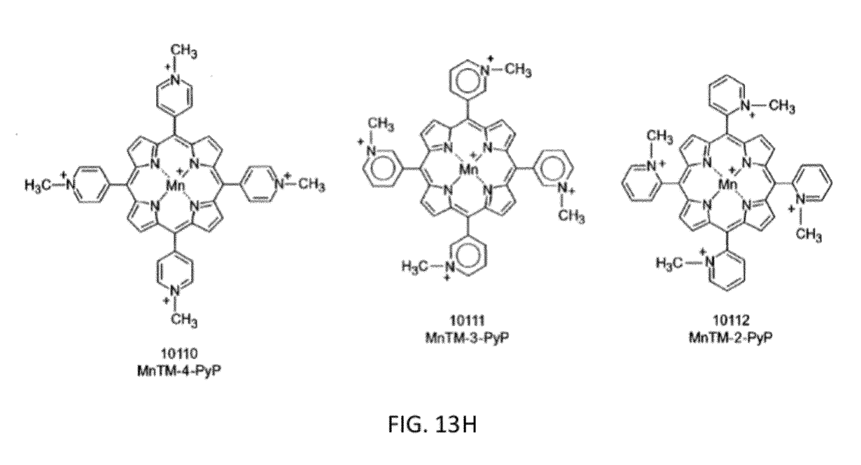

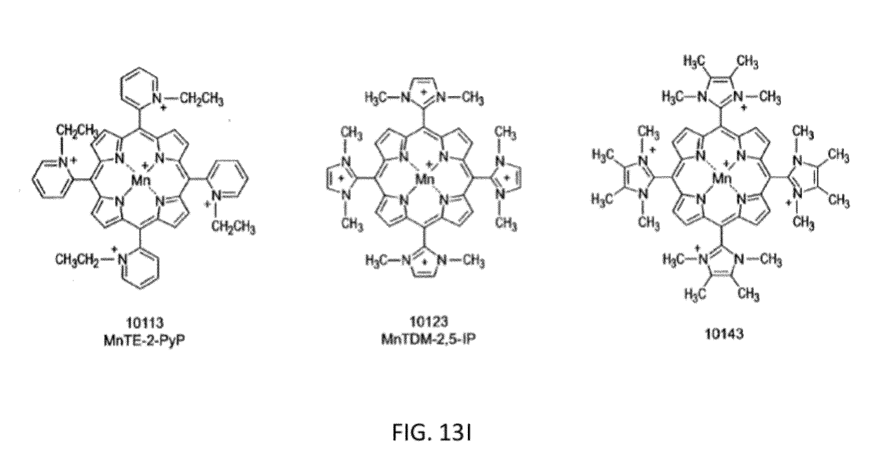

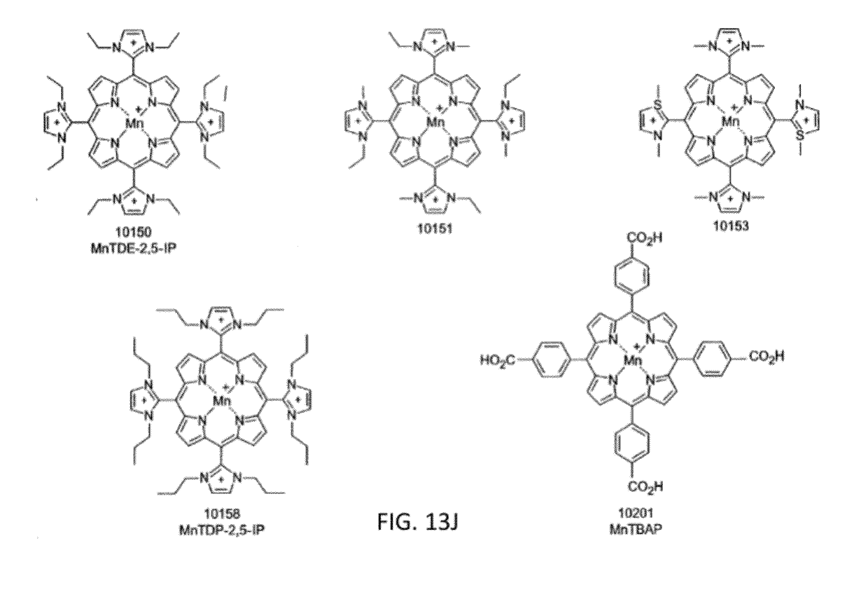

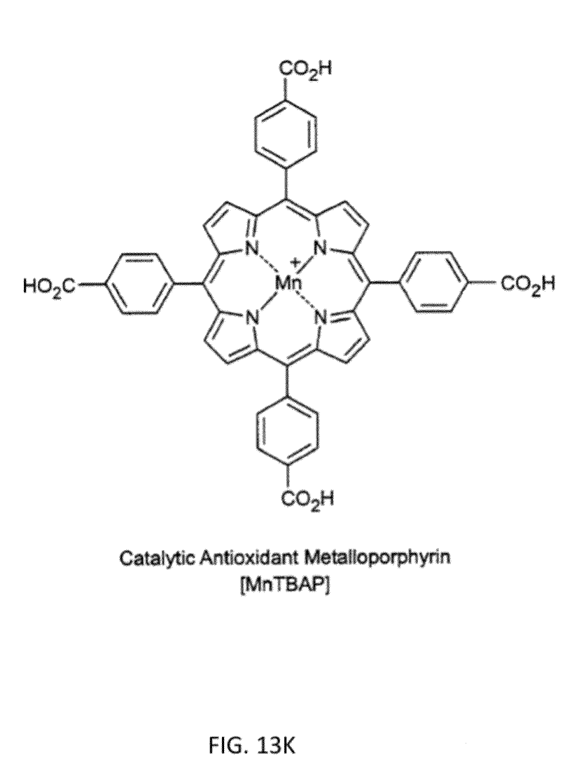

Disclosed herein are methods of maintaining stem cells in an undifferentiated state in vitro. The methods include contacting the stem cells with an effective amount of a catalytic antioxidant. Examples of catalytic antioxidants that can be used in the disclosed methods are those with a redox-active metal center that catalyzes the dismutation of O₂⁻. Examples of catalytic antioxidants appropriate for use in the present methods include methine (for example meso) substituted porphyrins and substituted tetrapyrroles, or pharmaceutically acceptable salts thereof. Both metal free and metal bound porphyrins and tetrapyrroles can be used in the disclosed methods. By way of example, it may be advantageous to add a metal free catalytic antioxidant to media that contains one or more metal ions. In the case of metal-bound porphyrins and tetrapyrroles, manganic derivatives are preferred, however, metals other than manganese such as iron (II or III), copper (I or II), cobalt (II or III), or nickel (I or II), can also be used. It will be appreciated that the metal selected can have various valence states, for example, manganese II, III, IV or V can be used. Zinc (II) can also be used even though it does not undergo a valence change and therefore do not directly scavenge superoxide. The choice of the metal can affect selectivity of the oxygen species that is scavenged. Particular examples of catalytic antioxidants are described in U.S. Patent Publication No. 2003/0032634, which is specifically incorporated herein by reference in its entirety (see for example FIG. 9 and FIG. 18, reproduced herein as FIGS. 1, 12 and 13). Thus, any of the catalytic antioxidants shown in FIGS. 1, 12 and 13 can be used in the disclosed methods.

Additional examples of catalytic antioxidants are described in U.S. Pat. Nos. 5,994,339, 6,103,714, 6,127,356, 6,479,477, 6,916,799, and 7,189,707 and International Patent Publication No. WO2010/009327, the disclosures of which are specifically incorporated herein by reference in their entirety to the extent that they disclose catalytic antioxidants and methods of producing catalytic antioxidants. In addition to the catalytic antioxidants described in the above identified patents and applications, other examples of catalytic antioxidants can also be used, including manganese salen compounds (Baudry et al., Biochem. Biophys. Res. Commun. 192:964 (1993)), manganese macrocyclic complexes, such as those described by Riley et al., (Inorg. Chem. 35:5213 (1996)), Deune et al., (Plastic Reconstr. Surg. 98:712 (1996)), Lowe et al., (Eur. J. Pharmacol. 304:81 (1996)) and Weiss et al., (J. Biol. Chem. 271:26149 (1996)), nitroxides (Zamir et al., Free Radic. Biol. Med. 27:7-15 (1999)), fullerenes (Lai et al., J. Autonomic Pharmacol. 17:229-235 (1997), Huang et al., Free Radic. Biol. Med. 30:643-649 (2001), Bensasson et al., Free Radic. Biol. Med. 29:26-33 (2000)), CuPUPY (Steinkuhler et al., Biochem. Pharmacol. 39:1473-1479 (1990)) and CuDIPS (Steinkuhler et al., Biochem. Pharmacol. 39:1473-1479 (1990)). (See also U.S. Pat. Nos. 6,084,093, 5,874,421, 5,637,578, 5,610,293, 6,177,419, 6,046,188, 5,834,509, 5,827,880, 5,696,109, and 5,403,834). In specific examples, the catalytic antioxidant is FBC-007, which is also referred to in the literature as AEOL 10113 (see e.g. U.S. Patent Publication No. 2003/0032634).

B. Methods of Inhibiting Differentiation of Stem Cells

Disclosed herein are methods for preventing or inhibiting differentiation of stem cells while maintaining the stem cells phenotype. The disclosed methods include contacting a stem cell, such as a pluripotent, multipotent or totipotent stem cell, during in vitro manipulation with an effective amount of a catalytic antioxidant that is capable of preventing or inhibiting the differentiation of the stem cell. Exemplary catalytic antioxidants for use in the disclosed methods are given in Section A above.

The inhibition of differentiation of stem cells does not require that all aspects of the stem cell phenotype be retained, but rather that at least one or more characteristics of that phenotype be retained (although not necessarily at the same level as native cells). For example, one or more of the following characteristics are retained: the expression of one or more surface marker or surface antigen; the level of expression of one or more surface marker or surface antigen; permeability to a histologic dye; morphology in culture; association with other cells in culture; and sensitivity to pharmacologic agents.

In some examples the stem cells are contacted with an effective amount of a catalytic antioxidant that is at a concentration between about 0.1 μM and about 500 μM, such as about 0.1 μM, 0.5 μM, 1 μM, about 2 μM, about 4 μM, about 6 μM, about 8 μM, about 12 μM, about 16 μM, about 18 μM, about 22 μM, about 26 μM, about 30 μM, about 34 μM, about 40 μM, about 50 μM, about 60 μM, about 70 μM, about 80 μM, about 90 μM, about 100 μM, about 150 μM, about 200 μM, about 250 μM, about 300 μM, about 350 μM, about 400 μM, or about 500 μM, such as between about 1 μM and about 10 μM, between about 5 μM and about 20 μM, between about 10 μM and about 40 μM, between about 30 μM and about 50 μM, between about 40 μM and about 100 μM, between about 50 μM and about 120 μM, between about 75 μM and about 200 μM, between about 100 μM and about 250 μM, between about 200 μM and about 400 μM, or between about 350 μM and about 500 μM. These ranges are illustrative only and it is contemplated that effective amounts or concentrations of the catalytic antioxidants could vary from these illustrative examples, for example higher or lower than what is given, for example depending the type of stem cell.

The disclosed methods are applicable to any in vitro manipulation of stem cells, including without limitation general cell culture in a research environment, in vitro processing in a clinical setting, or other setting where stem cells are produced for stem cell therapeutics.

In some example, the stem cells are contacted in vitro, for example after they are collected from a subject, such as a human subject. Methods of collecting stem cells are known to those of ordinary skill in the art. In some examples, the stem cells are totipotent stem cells. In some examples, stem cells are pluripotent stem cells. In some examples, the stem cells are multipotent stem cells. In some examples, the stem cells are mesenchymal stem cells, such as umbilical cord mesenchymal stem cells, for example human umbilical cord mesenchymal stem cells. In some examples, the stem cells are muscle stem cells, such as human muscle stem cells. In some examples, the stem cells are cancer stem cells, such as CD44hiCD24lo cancer stem cells, CD44hi cells are cells that express CD44 at a high level on their surface. Conversely CD24lo cells are cells that express CD24 at a low level on their surface. Using techniques such as flow cytometry subpopulations of CD44hiCD24lo cells can be isolated from other cells in a sample. Other markers can be used in the same way to isolate subpopulations of stem cells from a sample.

Methods are also disclosed herein for increasing the number of stem cells while maintaining the stem cells in an undifferentiated state. The number of stem cells can be increased by increasing proliferation of the cells. The methods include contacting the stem cells with an effective amount a catalytic antioxidant, such as those in Section A, and an effective amount of one or more growth factors in expansion medium that promotes the expansion of the stem cells. Growth factors and expansion media that promote the expansion of stem cells are known in the art. Using the methods described herein, it is possible to expand stem cells in culture without the cells losing the characteristics of stem cells such as the phenotypical differences that distinguish stem cells from other cells. In some examples, expansion of stem cells results in an increase of at least 10-fold in the number of stem cells without loss of the stem cell characteristics, for example at least a 50-fold increase, at least a 100-fold increase, at least a 150-fold increase, at least a 200-fold increase, at least a 250-fold increase, at least a 300-fold increase, at least a 400-fold increase, at least a 500-fold increase, at least a 1000-fold increase at least a 10000-fold increase, at least a 100000-fold increase or at least a 1,000,000-fold increase or even greater than 1,000,000 fold increase in the number of stem cells. In some examples, the stem cells are mesenchymal stem cells, such as umbilical cord mesenchymal stem cells, for example human umbilical cord mesenchymal stem cells. In some examples, the stem cells are muscle stem cells, such as human muscle stem cells. In some examples, the stem cells are cancer stem cells, such as CD44hi CD24lo cancer stem cells. In some examples, the expansion medium is supplemented with growth factors that promote the expansion of stem cells in culture. In some examples, the expansion medium is supplemented with one or more of epidermal growth factor (EGF), fibroblast growth factor 2 (FGF-2), insulin-like growth factor 1 (IGF-1), FLT-3 ligand, or stem cell factor (SCF). These growth factors have been demonstrated to stimulate proliferation of either myogenic precursor cells or stem cells. For example, FGF-2 and IGF-1 have been observed to stimulate proliferation of myogenic precursor cells and EGF and SCF have been shown to stimulate proliferation of stem cells in the hematopoietic compartment and central nervous system.

In several examples, the methods result in increased survival and/or increased proliferation of stem cells while maintaining the stem cells in an undifferentiated state. In some examples, the expanded stem cells are transplanted into a subject. In some examples, the stem cells are tested for phonotypic markers, such as cell surface markers to confirm that the stem cells have remained undifferentiated. Methods of characterizing stem cells by the presence or absence of cell surface markers are known in the art. In some examples, the stem cells are karyotyped to confirm that the stem cells have remained undifferentiated. Methods of karyotyping stem cells are known in the art. In some examples, the telomerase activity and/or telomere length is measured in the stem cells to confirm that the stem cells remain undifferentiated. Methods of measuring telomerase activity or telomere length are known in the art, for example using the TeloTAGGG Telomerase PCR ELISA PLUS kit (Roche) according to manufacturer's protocol or the cytometry-based measurement of telomere length PNA Kit for Flow Cytometry. In some examples, the expanded stem cells produced by the disclosed methods are used to screen compounds, such as drug for activity in the stem cells.

C. Stem Cells

Any stem cell can be used with the methods disclosed herein. For example, somatic stem cells can be treated as described by the methods disclosed herein, for example to expand a population of somatic stem cells without the somatic stem cells differentiation or losing the phenotypical characteristics that distinguish stem cells from other cells. The somatic stem cells can be isolated from a variety of sources using methods known to one skilled in the art. The somatic stem cells can be of ectodermal, mesodermal or endodermal origin. Any somatic stem cells which can be obtained and maintained in vitro can potentially be used in accordance with the present methods. Such cells include cells of epithelial tissues such as the skin and the lining of the gut, embryonic heart muscle cells, and neural precursor cells (Stemple and Anderson, Cell 71: 973-985 (1992)). Such cells also include pancreatic stem cells, cord blood stem cells, peripheral blood stem cells, and stem cells derived from adipose tissues. In some examples, the stem cells are muscle stem cells, such as human muscle stem cells. Human muscle cells are isolated using methods standard in the art (NDRI tissue) (see e.g. Qu-Petersen et al., J. Cell Biol. 157, 851-64 (2002); Lee et al., J. Cell Biol. 150, 1085-100 (2000); Rando & Blau, J. Cell Biol 125, 1275-87 (1994); Blau & Webster, Proc. Natl. Acad. Sci. U.S.A. 78, 5623-7 (1981); and Webster et al., Exp. Cell Res. 174, 252-65 (1988)).

Methods for isolating and culturing neuronal stem cells are disclosed, for example, in U.S. Pat. No. 6,610,540, which is incorporated herein by reference. Thus, methods are disclosed herein for increasing the number of neuronal stem cells, neuronal precursor cells and/or glial precursor cells. Mesenchymal progenitors give rise to a very large number of distinct tissues (Caplan, J. Orth. Res. 641-650, (1991)). Mesenchymal cells capable of differentiating into bone and cartilage have also been isolated from marrow (Caplan, J. Orth. Res. 641-650, (1991)). U.S. Pat. No. 5,226,914 describes an exemplary method for isolating mesenchymal stem cells from bone marrow. In some examples, the stem cells are mesenchymal stem cells, such as umbilical cord mesenchymal stem cells, for example human umbilical cord mesenchymal stem cells.

In other examples, the somatic stem cells are epithelial stem cells or keratinocytes can be obtained from tissues such as the skin and the lining of the gut by known procedures (Rheinwald, Meth. Cell Bio. 21A:229, (1980)). In stratified epithelial tissue such as the skin, renewal occurs by mitosis of precursor cells within the germinal layer, the layer closest to the basal lamina. Stem cells within the lining of the gut provide for a rapid renewal rate of this tissue.

The cells can also be liver stem cells (see PCT Publication No. WO 94/08598) or kidney stem cells (see Karp et al., Dev. Biol. 91:5286-5290, (1994)). The cells can also be inner ear stem cells (see Li et al., TRENDSMol. Med. 10: 309, 2004).

In some examples, the stem cells are cancer stem cells. For example, the compounds and methods disclosed herein can be used to inhibit the differentiation of cancer stem cells, such as cancer stem cells obtained from a tumor, such as a solid tumor or a tumor of the blood.

Examples of hematological tumors from which a cancer stem cell can be obtained include leukemias, such as acute leukemias, chronic leukemias, polycythemia vera, lymphoma, Hodgkin's disease, non-Hodgkin's lymphoma, multiple myeloma, Waldenstrom's macroglobulinemia, heavy chain disease, myelodysplastic syndrome, hairy cell leukemia, and myelodysplasia. Examples of solid tumors from which a cancer stem cell can be obtained include sarcomas and carcinomas, CNS tumors, and skin cancers. Illustrative cancer stem cells that may be targeted include, for example, but not by way of limitation, breast cancer, prostate cancer, glioblastoma, colon carcinoma, lung carcinoma, pancreatic cancer, melanoma, gastric cancer, hepatic carcinoma, ovarian carcinoma, and testicular cancer. Other cancer stem cells for targeting include lymphoma and leukemia.

The stability of the undifferentiated phenotype of cancer stem cells does not require that all aspects of the cancer stem cell phenotype be retained, but rather that at least one or more characteristics of that phenotype be retained (although not necessarily at the same level as native cells). In other words, the maintenance of cancer stem cells in an undifferentiated state does not require that all aspects of the cancer stem cell phenotype be retained. For example, one or more of the following characteristics are retained: the expression of one or more surface marker or antigen; the level of expression of one or more surface marker antigen; permeability to a histologic dye; number of cells required to produce a tumor when implanted into a host animal; characteristics of tumors produced from the cells; morphology in culture; association with other cells in culture; and sensitivity to pharmacologic agents.

A cancer stem cell obtained from any type of cancer may be maintained in an undifferentiated state that is phenotype of the cancer cell may be stabilized, for example maintained. The cancer stem cell may be from a human or a non-human subject. The cancer stem cell may be obtained from a tumor cell line or for a primary tumor.

A cancer stem cell may be collected by any means known in the art. For example, a cancer stem cell may be collected from (isolated from or enriched from) a larger population of cells using cell surface markers or other properties typical to that cancer stem cell. Alternatively, an Oct3/4 promoter sequence-containing construct which contains a selectable marker which is selectively expressed in a cancer stem cell may be collected from the population via the selectable marker, for example by fluorescence activated cell sorting (“FACS”). The preparation and use of immortalized cancer stem cells is described in PCT International Publication No. WO 2009/140260, “Cancer Stem Cell Immortalization”, filed May 12, 2009, which is incorporated herein by reference in its entirety. For example, the introduction of an Oct3/4 promoter sequence was observed to stabilize the undifferentiated phenotype of cancer stem cells. This effect is alternatively referred to herein as “immortalization”, which as defined herein, does not require that a culture of such cells would persist indefinitely.

Expression of the ALDH1 isoform (9a) may be used as a cancer stem cell marker. For example, the Aldefluor Assay (Stem Cell Technologies, Inc.) may be used.

In non-limiting embodiments, where the cancer stem cell is a breast cancer stem cell, a phenotype of cell marker expression CD44hi (meaning increased relative to normal control) and CD24lo (meaning decreased relative to normal control) may be used to collect cancer stem cells (for example, using antibodies directed to said proteins and FACS). Where a cell line is used as the source of cancer stem cells, suitable cell lines include, but are not limited to MCF7, T-47D, UACC-812, HCC38, HCC1428, SKBR-3, and MB-157.

In non-limiting embodiments, where the cancer stem cell is a colon cancer stem cell, a phenotype of cell marker expression EpCAMhi/CD44hi, or expression of CD133, or the ability to exclude the dye Hoechst 33342, may be used to collect cancer stem cells. Where a cell line is used as the source of cancer stem cells, suitable cell lines include, but are not limited to Colo320, HCT15, and SW480.

In non-limiting embodiments, where the cancer stem cell is a prostate cancer stem cell, a phenotype of cell marker expression CD44hiCD24lo/Scal+ or the ability to exclude the dye Hoechst 22243, may be used to collect cancer stem cells. Where a cell line is used as the source of cancer stem cells, suitable cell lines include, but are not limited to PC3, DU145, and LNCaP.

In non-limiting embodiments, where the cancer stem cell is a pancreatic cancer stem cell, a phenotype of cell marker expression CD44hi, CD24hi, ESAhi may be used to collect cancer stem cells. Where a cell line is used as the source of cancer stem cells, suitable cell lines include, but are not limited to PANC-1 and ASPC-1.

In some examples, the stem cells are embryonic stem cells. For example, murine, primate or human embryonic stem cells can be utilized. In several examples, the cells are embryonic stem (ES) cells, which can proliferate indefinitely in an undifferentiated state. Furthermore, ES cells are totipotent cells, meaning that they can generate all of the cells present in the body (bone, muscle, brain cells, etc.). ES cells have been isolated from the inner cell mass (ICM) of the developing murine blastocyst (Evans et al, Nature 292: 154-156, 1981; Martin et al., Proc. Natl. Acad. ScL 78:7634-7636, 1981; Robertson et al., Nature 323:445-448, 1986). Additionally, human cells with ES properties have been isolated from the inner blastocyst cell mass (Thomson et al., Science 282: 1145-1147, 1998) and developing germ cells (Shamblott et al., Proc. Natl. Acad. Sci. USA 95: 13726-13731, 1998), and human and non-human primate embryonic stem cells have been produced (see U.S. Pat. No. 6,200,806, which is incorporated by reference herein).

As disclosed in U.S. Pat. No. 6,200,806, ES cells can be produced from human and non-human primates. In one embodiment, primate ES cells are isolated “ES medium” that express SSEA-3; SSEA-4, TRA-1-60, and TRA-1-81 (see U.S. Pat. No. 6,200,806). ES medium consists of 80% Dulbecco's modified Eagle's medium (DMEM; no pyruvate, high glucose formulation, Gibco BRL), with 20% fetal bovine serum (FBS; Hyclone), 0.1 mM β-mercaptoethanol (Sigma), 1% non-essential amino acid stock (Gibco BRL). Generally, primate ES cells are isolated on a confluent layer of murine embryonic fibroblast in the presence of ES cell medium. In one example, embryonic fibroblasts are obtained from 12 day old fetuses from out bred mice (such as CF1, available from SASCO), but other strains may be used as an alternative. Tissue culture dishes treated with 0.1% gelatin (type I; Sigma) can be utilized. Distinguishing features of ES cells, as compared to the committed “multipotential” stem cells present in adults, include the capacity of ES cells to maintain an undifferentiated state indefinitely in culture, and the potential that ES cells have to develop into every different cell types. Unlike mouse ES cells, human ES (hES) cells do not express the stage-specific embryonic antigen SSEA-I, but express SSEA-4, which is another glycolipid cell surface antigen recognized by a specific monoclonal antibody (see, for example, Amit et al, Devel. Biol. 227:271-278, 2000).

Cell lines may be karyotyped with a standard G-banding technique (such as by the Cytogenetics Laboratory of the University of Wisconsin State Hygiene Laboratory, which provides routine karyotyping services) and compared to published karyotypes.

Human ES cell lines exist and can be used in the methods disclosed herein. Human ES cells can also be derived from preimplantation embryos from in vitro fertilized (IVF) embryos. Studies on unused human IVF-produced embryos are allowed in many countries, such as Singapore and the United Kingdom, if the embryos are less than 14 days old. Only high quality embryos are suitable for ES isolation. Present defined culture conditions for culturing the one cell human embryo to the expanded blastocyst have been described (see Bongso et al., Hum Reprod. 4:706-713, 1989). Co-culturing of human embryos with human oviductal cells results in the production of high blastocyst quality. IVF-derived expanded human blastocysts grown in cellular co-culture, or in improved defined medium, allows isolation of human ES cells with the same procedures described above for non-human primates (see U.S. Pat. No. 6,200,806).

D. Exemplary Compositions of Expanded Stem Cells

Expanded populations of stem cells produced from the methods described herein may be used for the formulation of pharmaceutical or non-pharmaceutical compositions. As discussed herein, these formulations are useful in a variety of therapeutic and research applications.

In some embodiments, the pharmaceutical composition includes one or more stem cells produced from the methods described herein and a pharmaceutically acceptable carrier. In various embodiments, the cells are isolated or purified. While any suitable carrier known to those of ordinary skill in the art may be employed in the pharmaceutical compositions of this disclosure, the type of carrier will vary depending on the mode of administration. Compositions can be formulated for any appropriate manner of administration, including, for example, oral, intravenous, intra-arterial, intravesicular, inhalation, intraperitoneal, intrapulmonary, intramuscular, subcutaneous, intra-tracheal, transmucosal, intraocular, intrathecal, or transdermal administration. For parenteral administration, such as subcutaneous injection, the carrier may include, e.g., water, saline, alcohol, a fat, a wax, or a buffer. Biodegradable microspheres (e.g., polylactate polyglycolate) may also be used as carriers. In some embodiments, cells are administered directly into a tissue or organ, such as the bone marrow, brain, liver, kidney, pancreas, spleen, or other parenchymal organs.

In some embodiments, the pharmaceutical or non-pharmaceutical compositions include a buffer (e.g., neutral buffered saline, phosphate buffered saline, etc), a carbohydrate (e.g., glucose, mannose, sucrose, dextran, etc), an antioxidant, a chelating agent (e.g., EDTA, glutathione, etc.), a preservative, another compound useful for treating a condition, an inactive ingredient (e.g., a stabilizer, filler, etc), or combinations of two or more of the foregoing.

The compositions described herein may be administered as part of a sustained release formulation (e.g., a formulation such as a capsule or sponge that produces a slow release of cells following administration). In some embodiments, the cells are released over a period of about any of 4 hours, 8 hours, 12 hours, 16 hours, 1 day, 2 days, 3 days, 4 days, 5 days, 6 days, 1 week, 2 weeks, or more. In some embodiments, at least about any of 10%, 15%, 20%, 25%, 30%, 40%, 50%, 60%, 65%, 70%, 75%, 80%, 90%, 95%, 99%, or 100% of the released cells are viable. Such formulations may generally be prepared using well known technology and administered by, for example, rectal or subcutaneous implantation, or by implantation at the desired target site. Sustained-release formulations may contain cells dispersed in a carrier matrix and/or contained within a reservoir surrounded by a rate controlling membrane. Carriers for use within such formulations are biocompatible, and may also be biodegradable. In some embodiments, the formulation provides a relatively constant level of cell release. The amount of cells contained within a sustained release formulation depends upon the site of implantation, the rate and expected duration of release, and the nature of the condition to be treated or prevented.

It will be appreciated that the unit content of active ingredients contained in an individual dose of each dosage form need not in itself constitute an effective amount since the necessary effective amount could be reached by the combined effect of a plurality of administrations. The selection of the amount of cells to include in a pharmaceutical composition depends upon the dosage form utilized, the condition being treated, and the particular purpose to be achieved according to the determination of the ordinarily skilled artisan in the field.

To reduce or prevent an immune response in human subjects who are administered a pharmaceutical composition, the pharmaceutical composition may also include one or more immunosuppressive agents, such as cyclosporin. To bias the cells towards a desired cell type, the pharmaceutical composition may also include one or more growth factors, hormones, interleukins, cytokines, NGF, or other cells.

Methods are provided for the treatment or prevention of disease in an individual (e.g., a mammal, such as a primate (e.g., a human, a monkey, a gorilla, an ape, a lemur, etc) that include administering one or more stem cells produced from the methods described herein to the individual. For example, an effective amount includes one or more stem cells produced from the methods described herein that can be administered to an individual in need of one or more cell types to treat a disease, disorder, or condition.

Examples of diseases, disorders, or conditions that may be treated or prevented include neurological, endocrine, structural, skeletal, vascular, urinary, digestive, integumentary, blood, immune, auto-immune, inflammatory, endocrine, kidney, bladder, cardiovascular, cancer, circulatory, digestive, and muscular diseases, disorders, and conditions. Since many human diseases result from defects in a single cell type, replacing defective cells by cell or tissue replacement therapy using stem cells can alleviate the symptoms of or cure various degenerative diseases. For example, the stem cells produced from the methods described herein can be differentiated into cells such as pancreatic beta cells to treat diabetes or differentiated into cells such as substantia nigral dopaminergic neuronal cells to treat Parkinson's disease and muscle cells to treat Duchenne Muscular Dystrophy. Exemplary hematopoietic conditions include blood and immune conditions. In some embodiments, these cells are used for reconstructive applications, such as for repairing or replacing tissues or organs. Other exemplary conditions include diseases of reproductive organs, skin, wound healing, and cosmetic conditions (such as hair loss, nails, etc).

With respect to the therapeutic methods described herein, it is not intended that the administration of stem cells produced from the methods described herein to an individual be limited to a particular mode of administration, dosage, or frequency of dosing; the present invention contemplates all modes of administration, including intramuscular, intravenous, intraarticular, intralesional, subcutaneous, or any other route sufficient to provide a dose adequate to treat a condition. Both systemic and local administration is contemplated. The cells may be administered to the individual in a single dose or multiple doses. When multiple doses are administered, the doses may be separated from one another by, for example, one week, one month, one year, or ten years. One or more growth factors, hormones, interleukins, cytokines, small molecules, peptides, antibodies, or other cells may also be administered before, during, or after administration of the cells to further bias them towards a particular cell type. Additionally, one or more immunosuppressive agents, such as cyclosporin, may be administered to inhibit rejection of the transplanted cells. It is to be understood that, for any particular subject, specific dosage regimes should be adjusted over time according to the individual need and the professional judgment of the person administering or supervising the administration of the compositions.

E. Exemplary Uses of Stem Cells for Research and Drug Screening Applications

The stem cells produced from the methods described herein can be used in a variety of research and drug screening applications. For example, these cells can be used to determine the effect of candidate compounds on cell division (e.g., meiosis or mitosis), chromosome behavior, recombination (e.g., homologous recombination), genomic imprinting, self-renewal, differentiation, maturation, migration, or any two or more of the foregoing. In some embodiments, stem cells produced from the methods described herein are contacted with a candidate compound, and cell division, chromosome behavior, recombination, genomic imprinting, self-renewal, differentiation, maturation, or migration, or any two or more of the foregoing is measured or assayed. The candidate compound is determined to modulate cell division, chromosome behavior, recombination, genomic imprinting, self-renewal, differentiation, maturation, or migration if the candidate compound causes a change in cell division, chromosome behavior, recombination, genomic imprinting, respectively. Cell proliferation can be studied by evaluating the cell cycle using fluorescence-activated cell-sorting (FACS). Mechanisms regulating self-renewal of stem cells and the differentiation of stem cells can be studied using standard methods. Compounds and incubation conditions can also be tested to determine conditions that induce the differentiation of stem cells, including the differentiation of cancer stem cells. Mechanisms that regulate migration of stem cells and their derivatives are also important for transplantation strategies. Numerous compounds can be tested, such as peptide libraries, antibody libraries, small molecule libraries, etc. These approaches can be useful for all aspects of regenerative medicine.

In particular examples, cancer stem cells that have been phenotypically stabilized may be used to identify useful therapeutic agents, by screening various test agents. The test agents may be known bioactive compounds or may be compounds without hitherto known biological activity. Suitable test agents may also be biological molecules, including but not limited to proteins, antibodies or antibody fragments, oligonucleotides, peptidomimetic compounds, and additional agents. An exemplary method of identifying an anti-cancer agent includes providing an isolated cancer stem cell that is maintained in an undifferentiated state and contacting the stem cell with a suitable test compound. The proliferation and/or differentiation and/or viability of the cancer stem cell is evaluated wherein an inhibition of proliferation, increase in level of differentiation, or decrease in viability associated with the presence of the test agent indicates that the test agent is an anti-cancer agent. In certain non-limiting embodiments of this method, the means for evaluating the proliferation, differentiation level, and/or viability comprise measuring and/or detecting expression of a reporter gene.

Exemplary test agents that can be screened include, but are not limited to, peptides such as, soluble peptides, including but not limited to members of random peptide libraries (see, e.g., Lam et al., Nature, 354:82-84, 1991; Houghten et al., Nature, 354:84-86, 1991), and combinatorial chemistry-derived molecular library made of D- and/or L-configuration amino acids, phosphopeptides (including, but not limited to, members of random or partially degenerate, directed phosphopeptide libraries; see, e.g., Songyang et al., Cell, 72:767-778, 1993), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), small organic or inorganic molecules (such as, so-called natural products or members of chemical combinatorial libraries), molecular complexes (such as protein complexes), or nucleic acids.

Appropriate agents can be contained in libraries, for example, synthetic or natural compounds in a combinatorial library. Numerous libraries are commercially available or can be readily produced; means for random and directed synthesis of a wide variety of organic compounds and biomolecules, including expression of randomized oligonucleotides, such as antisense oligonucleotides and oligopeptides, also are known. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Such libraries are useful for the screening of a large number of different compounds.

Libraries of agents to be screened (such as combinatorial chemical libraries) useful in the disclosed methods include, but are not limited to, peptide libraries (see, e.g., U.S. Pat. No. 5,010,175; Furka, Int. J. Pept. Prot. Res., 37:487-493, 1991; Houghton et al., Nature, 354:84-88, 1991; PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Natl. Acad. Sci. USA, 90:6909-6913, 1993), vinylogous polypeptides (Hagihara et al., J. Am. Chem. Soc., 114:6568, 1992), nonpeptidal peptidomimetics with glucose scaffolding (Hirschmann et al., J. Am. Chem. Soc., 114:9217-9218, 1992), analogous organic syntheses of small compound libraries (Chen et al., J. Am. Chem. Soc., 116:2661, 1994), oligocarbamates (Cho et al., Science, 261:1303, 1003), and/or peptidyl phosphonates (Campbell et al., J. Org. Chem., 59:658, 1994), nucleic acid libraries (see Sambrook et al. Molecular Cloning, A Laboratory Manual, Cold Springs Harbor Press, N.Y., 1989; Ausubel et al., Current Protocols in Molecular Biology, Green Publishing Associates and Wiley Interscience, N.Y., 1989), peptide nucleic acid libraries (see, e.g., U.S. Pat. No. 5,539,083), antibody libraries (see, e.g., Vaughn et al., Nat. Biotechnol., 14:309-314, 1996; PCT App. No. PCT/US96/10287), carbohydrate libraries (see, e.g., Liang et al., Science, 274:1520-1522, 1996; U.S. Pat. No. 5,593,853), small organic molecule libraries (see, e.g., benzodiazepines, Baum, C&EN, January 18, page 33, 1993; isoprenoids, U.S. Pat. No. 5,569,588; thiazolidionones and methathiazones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514) and the like.

Libraries of agents useful for the disclosed screening methods can be produced in a variety of manners including, but not limited to, spatially arrayed multipin peptide synthesis (Geysen, et al., Proc. Natl. Acad. Sci., 81(13):3998-4002, 1984), “tea bag” peptide synthesis (Houghten, Proc. Natl. Acad. Sci., 82(15):5131-5135, 1985), phage display (Scott and Smith, Science, 249:386-390, 1990), spot or disc synthesis (Dittrich et al., Bioorg. Med. Chem. Lett., 8(17):2351-2356, 1998), or split and mix solid phase synthesis on beads (Furka et al., Int. J. Pept. Protein Res., 37(6):487-493, 1991; Lam et al., Chem. Rev., 97(2):411-448, 1997). Libraries may include a varying number of compositions (members), such as up to about 100 members, such as up to about 1000 members, such as up to about 5000 members, such as up to about 10,000 members, such as up to about 100,000 members, such as up to about 500,000 members, or even more than 500,000 members.

In one convenient embodiment, high throughput screening methods involve providing a combinatorial chemical or peptide library containing a large number of potential therapeutic compounds. Such combinatorial libraries are then screened in one or more assays as described herein to identify those library members (particularly chemical species or subclasses) that display a desired characteristic activity. The compounds identified using the methods disclosed herein can serve as conventional “lead compounds” or can themselves be used as potential or actual therapeutics. In some instances, pools of candidate agents may be identify and further screened to determine which individual or subpools of agents in the collective have a desired activity.

The disclosure is illustrated by the following non-limiting Examples.

### EXAMPLES

### Example 1

### Study of In Vitro and In Vivo Stem Cell Populations

This example provides exemplary procedures for investigating stem cell transplantation and differentiation.

Many studies of muscle stem cells for cell therapy for Duchene Muscular Dystrophy or muscle repair are currently using mouse muscle stem cells to address key mechanistic questions. In order to translate the work of these investigations, it is important to apply the findings in murine cells to human cells.

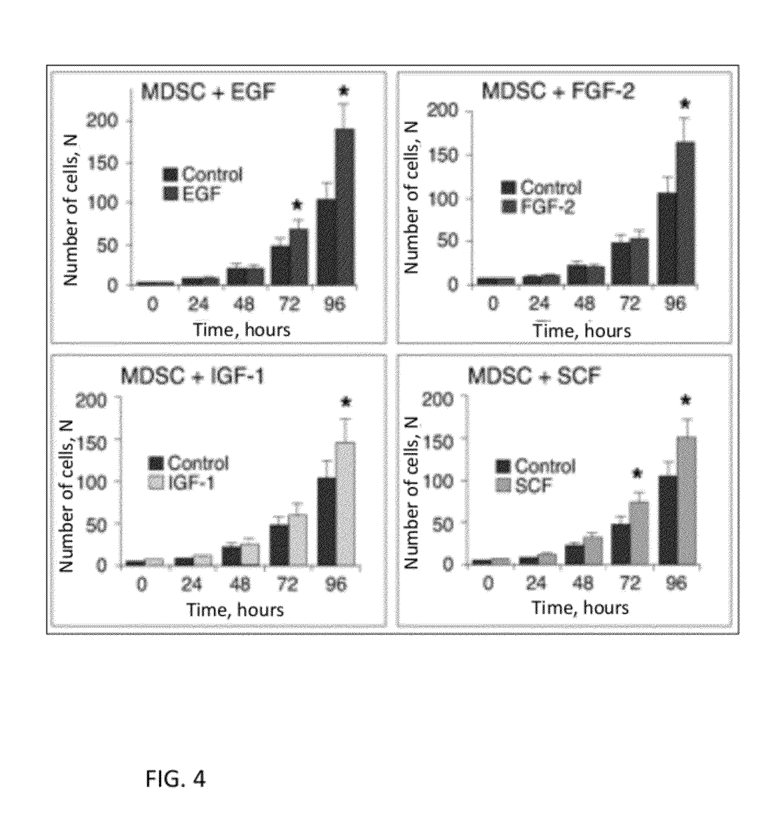

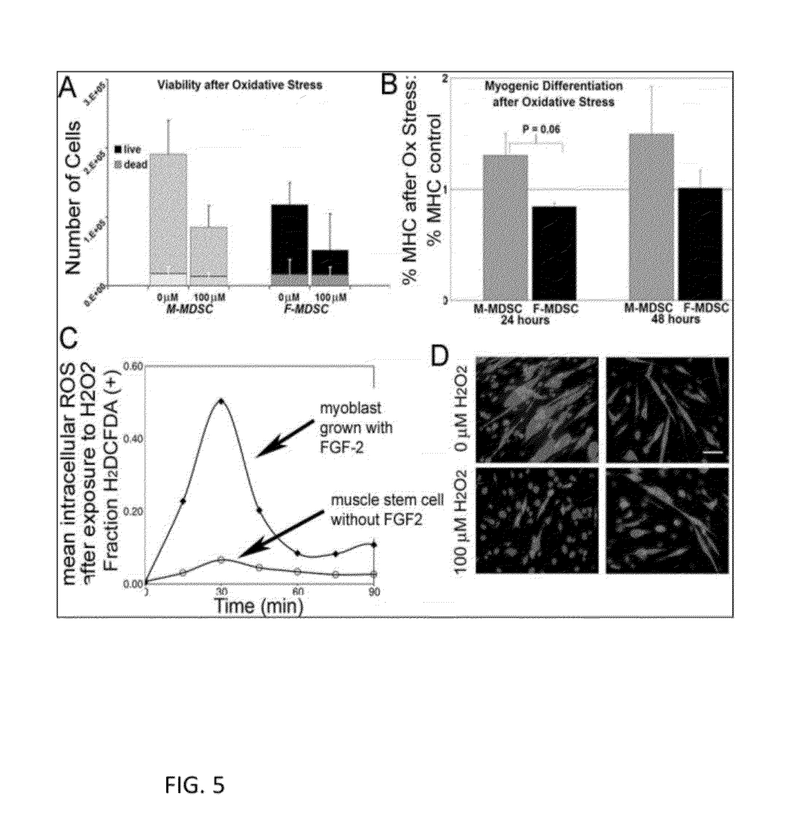

Previously several growth factors (GFs) were screened for their ability to stimulate mouse muscle stem cells in in vitro expansion. For example, as reported by Deasy et al., (Stem Cells 20, 50-60 (2002)), 6 GFs were tested: epidermal growth factor (EGF, 100 ng/ml), basic fibroblast growth factor (FGF-2, 100 ng/ml), insulin-like growth factor-1 (IGF-1, 100 ng/ml), FLT-3 ligand (25 ng/ml), hepatocyte growth factor (HGF, 25 ng/ml), and stem cell factor (SCF, 25 ng/ml). These growth factors had previously been demonstrated to stimulate proliferation of either myogenic precursor cells or stem cells. FGF-2 and IGF-1 and have been observed to stimulate proliferation of myogenic precursor cells. EGF and SCF have been shown to stimulate proliferation of stem cells in the hematopoietic compartment and central nervous system. Muscle cell population size, N, was increased significantly (p<0.05) with EGF, and also, to a lesser extent, by FGF-2, IGF-1 (p<0.05) and SCF (see FIG. 4). In studying these systems, the focus is on the changes in intracellular ROS levels and the oxidative damage after extended in vitro aging.

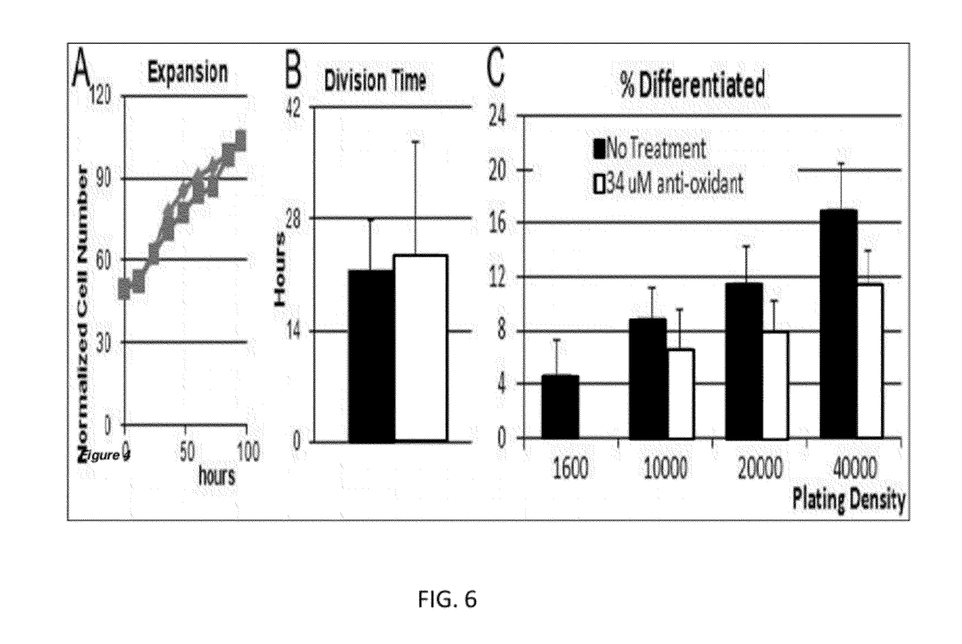

It was found that there was a high level of oxidative stress in both in vitro and in vivo stem cell populations. It was observed that oxidative stress lead to reduced cell viability and impairment of myogenic differentiation capacity of different populations of muscle stem cells. Initial results demonstrated that FBC-007 did not affect cell growth (FIGS. 6A and 6B), and prevented uninduced myogenic differentiation in the absence of GF stimulation (FIG. 6C).

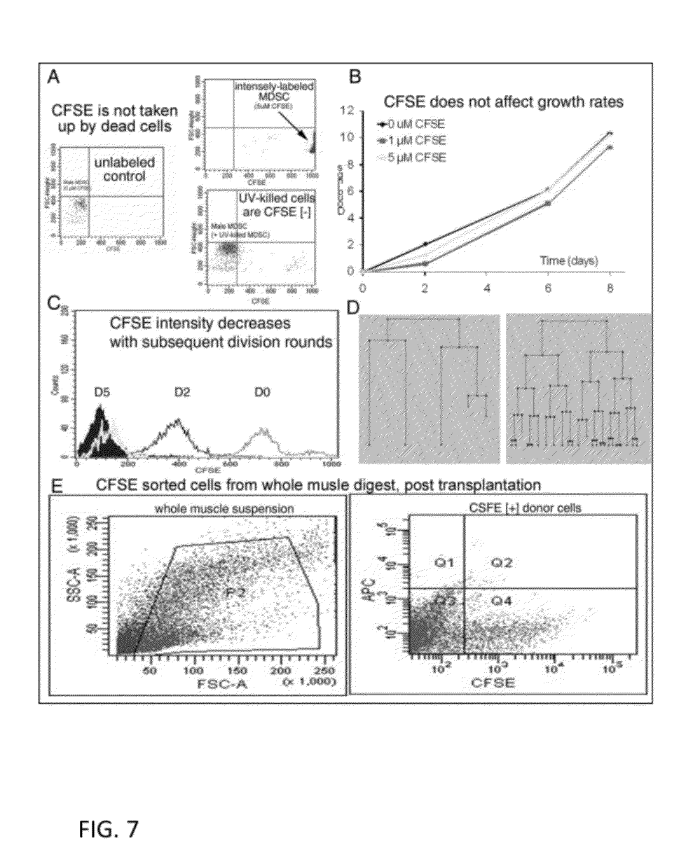

It was then determined that a quiescent subpopulation within mouse and human muscle stem cells is altered with GF-stimulation. This population was detected by several methods, including 1) direct observation and construction of cell lineage trees using live cell imaging (FIG. 7), 2) via (absence of) BrdU uptake, 3) with CFSE (FIG. 7) and 4) by estimation with the Sherley equation.

After stimulation of both mouse primary muscle cells and in vitro expanded cells with growth factors, it was observed that primary populations resulted in increased cell numbers by recruiting or activating nondividing cells. These studies used the Sherley equation to estimate the nondividing fraction based on data obtained from time-lapsed images. In addition, this technology was also used to validate the equation's assumptions, and derive new models based on the presence of nondividing cells and differentiated and apoptotic cells.

More recent studies have directly analyzed time-lapsed images and generated cell lineage trees. This provided the most direct and accurate confirmation that quiescent cells are present in the population. It was also confirmed that this quiescent stem cell population had the ability to re-enter the cell cycle through both symmetric and asymmetric divisions (FIG. 7D).

Nondividing cells were also detected within the human muscle cell population in the context of GF-dose-dependent studies. Using the time-lapsed imaging system, it was identified that there was a dose-dependent response of human skMSC to IGF-1 and FGF-2. There were significantly more muscle stem cells after 3 days in 100 ng/mL IGF-1 or basic FGF as compared to lower doses and unstimulated controls. The cells were pulsed with BrdU for 36 hours (division time=19±4 hrs) immunostaining was performed to detect labeled and unlabeled cells. It was found that there were significantly fewer nondividing cells (BrdU [−]) after only 3 days exposure to IGF-1 (P=0.01). These results demonstrated that between 26-50% of cells in the skMSCs are quiescent/nondividing.

Both time-lapsed imaging and FACS sorting were applied to the quiescent population using used cytoplasmic tracking dyes, used 5-6-carboxyfluorescein diacetate, succinimdyl ester, CFSE, and a similar molecule, CMFDA. Once cell permeable CFDA-SE is taken up by cells, intracellular esterases in the cytoplasm cleave acetate groups and convert CFDA to fluorescent CFSE. It was first confirmed that CFSE is impermeable; it was not taken in by unlabeled neighboring cells when CFSE-positive cells die (FIG. 7A). An approach was developed to use CFSE as an effective method for labeling of muscle cells. Concentrations subtending 5.0 μM showed muscle stem cells are similar in growth rate following CFSE treatment (FIG. 7A). During division, the relative fluorescent intensity of the cells is decreased in half; cells which do not divide retain a high level of CFSE (green) fluorescence (FIG. 7C).

Human skMSCs were separated by FACS into CMFDA[+] cells (nondividing) and CFMDA [−] (dividing cells) 9 days after labeling. A small subpopulation of nondividing cells relative to the total population was detected. Both subpopulations were subsequently analyzed by time-lapsed imaging. The quiescence of these CMFDA[+] cells was confirmed by examining growth rates and constructing cell lineage trees (FIG. 7D). It was observed that there was a high proliferation in the CFMDA [−] population. In comparison, most cells in the CMFDA [−] population did not divide or undergo cell death; but they did become activated and initiate divisions after an initial lag in growth, and this would be consistent with the expectation that quiescent cells should re-enter the cell cycle upon injury or to re-establish the population.

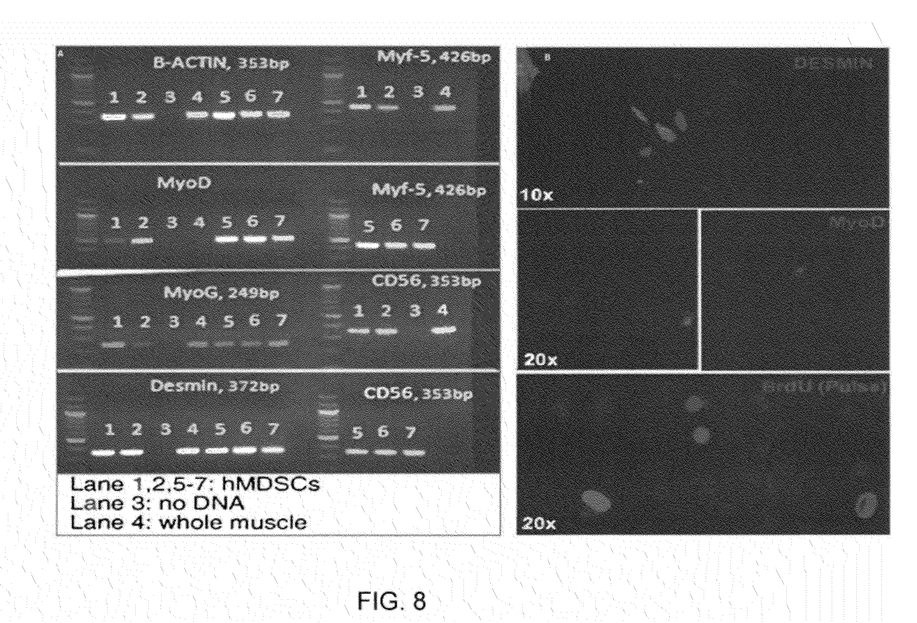

The established preplate technique was used to obtain human muscle stem cells from skeletal muscle biopsies, based on serial replating of low-adherent cells. The “slow adhering”, cells express CD56, but do not express CD34 or CD144 by flow analysis. skMSCs also expressed adhesion cell surface markers and mesenchymal markers, CD44, CD73, CD90, CD146 and CD105. Myogenic markers were detected by PCR and immune-staining (FIG. 8). Several phenotype changes could be involved with GF-induced cell culture aging of skMSCs. For example, recent reports showed a decline in myf5 or myoD is associated with aged muscles, and this may be also be associated with tissue fibrosis. A decline in myogenic marker expression, such as myoD, may also occur with GF expansion and in vitro culture-induced cell aging.

FIG. 9 illustrates the progression of differentiation in human skMSCs as cells express desmin, myosin heavy chain and even dystrophin in vitro. Human myogenic cells could be transplanted into the skeletal muscle of different animal models and it was possible to detect both human spectrin and dystrophin. After transplanting human myo-endothelial cells to injured skeletal muscle of SCID mice, fluorescent staining of human spectrin in the fibers could be seen one month after transplantation into the gastrocnemius muscle. This demonstrates the feasibility of cell transplantation in the mice and the specificity of the spectrin antibody (FIG. 9B). After transplantation of preplated human skMSCs, it was observed that there were significantly more dystrophin positive fibers as compared to PBS controls (FIG. 9C). Further, the donor cells can be identified by using a number of markers, including human lamin A/C or human chromosomal markers.

In regards to human skMSC expansion, initiation with 10⁵ cells can yield more than 10⁸ cells, or 670 million cells per sample, after 3.5 weeks of cell culture (n=3 human samples). Numerically, these numbers are sufficient for laboratory studies and transplantation to mice, but not sufficient for regeneration of >600 large human muscles, and stimulated expansion methods is necessary for clinical approaches.

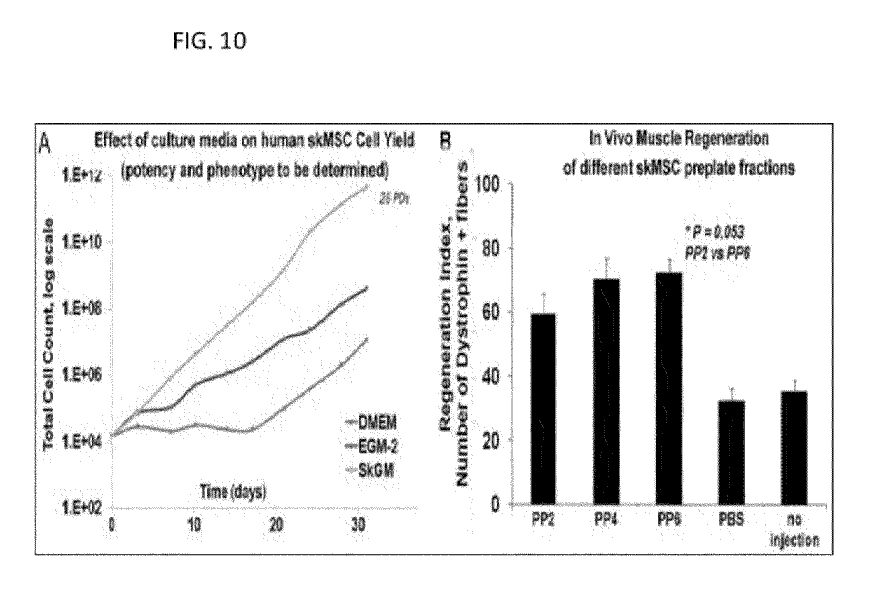

It is worth noting that whereas mouse muscle stem cell populations have been expanded to beyond 200 population doublings (PDs), the results to date show that human skMSCs will reach proliferative decline much sooner than mouse muscle cells when grown in the same medium (DMEM). The effect of 3 different culture media on the preplate derived human muscle cell populations was further studied. In this case, the greatest expansion potential was observed with SKGM (FIG. 10A), and not EGM2 which was previously showed favored human myoendothelial.

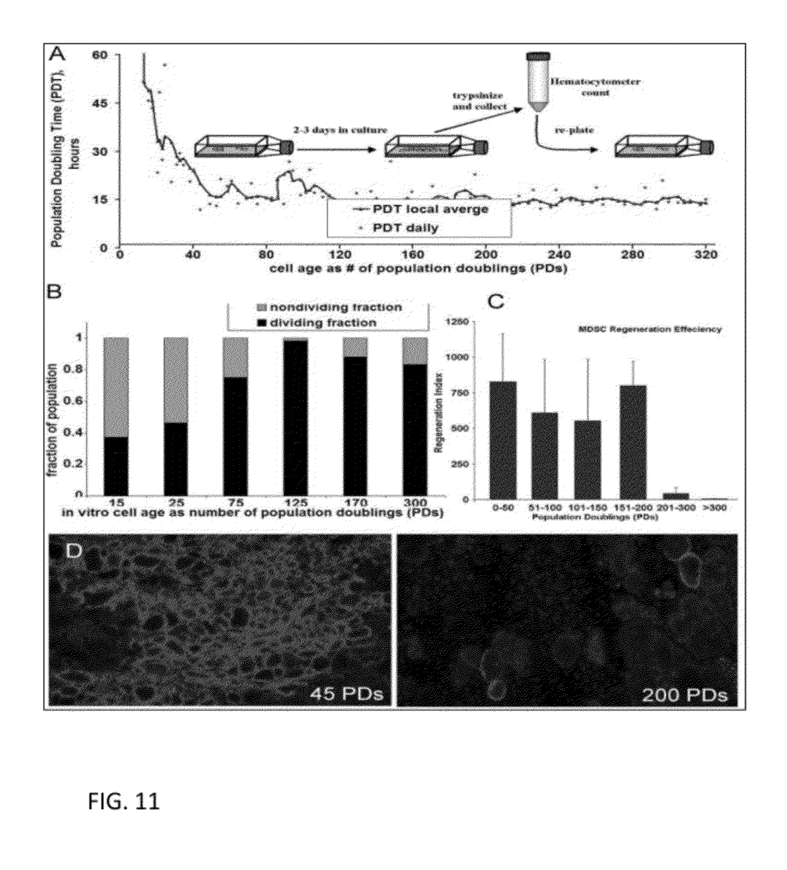

It was further observed that in vitro expansion reduced the number of dystrophin positive fibers present in the muscle after mouse cell transplants to dystrophic mdx animals (FIG. 11). Though these studies did not involve GF-stimulation, they illustrate the importance of identifying the point at which in vitro aging leads to detrimental changes. It was also observed that reduction of the quiescent/nondividing pool may be associated with reduced cell efficacy to participate in in vivo skeletal muscle regeneration after transplantation to mdx animals (FIGS. 11B and 11C). It was also observed that there was increased population growth and a decrease in the non-dividing fraction after expansion (from 63%—shortly after cell isolation (15 PDs)—to 25% to 2% after 75 PDs, FIG. 11C). A decrease in regeneration index (RI) was observed for mouse muscle cells after transplantation of 10⁵ cells. The RI for cells of 0-50 PDs was 829±337 dystrophin-positive fibers, for up to 195 PDs RI=800±170; but engraftment efficiency decreased significantly for cells at 200-300 PDs, R32±47 or cells >300 PDs, RI=3±3, P<0.001⁸⁹.

Expansion of mouse muscle stem cells beyond 200 PDs resulted in several changes in phenotypic markers, including loss of CD34 expression, loss of myogenic activity, reduced response to low serum, and increased growth on soft agar. Mouse muscle stem cells were transplanted subcutaneously into SCID mice to examine the cells' neoplastic growth potential. Cells that we had expanded to 30 PDs, or 300 PDs formed neoplastic growths in 0 of 8, and 1 of 8 injection sites, respectively.

### Example 2

### In Vitro Expansion Induces Stem Cell Aging, which is Reduced with Anti-Oxidant Treatment

This example demonstrates that age related changes in stems cells, as exemplified by muscle stem cells, are due to damage from reactive oxygen species (ROS). The example further demonstrates that the damage can be reduced or halted using catalytic antioxidants. While this example references muscle stem cells, it is equally applicable to other stems cell types that undergo differentiation.

To demonstrate that age-related changes in stem cells are due, in part, to the increase in reactive oxygen species (ROS) generated upon growth factor stimulation, and subsequent accumulation of oxidative DNA damage, stem cell populations are expanded in the presence and absence of growth factors (GFs) and the rate of accumulation of ROS, and oxidative DNA damage is examined. Throughout the course of the expansion, the level of mitigators of oxidative stress, such as superoxide dismutase, glutathione peroxidase, catalase and glutathione is also measured. The telomere length and telomerase activity of the stem cells is also measured to determine the velocity of change, for the purpose of determining relative change in the age of the stem cells. In addition, the numerical limit of the cell expansion is determined, and the clinical limit or point in expansion at which the stem cells show signs of in vitro aging is determined.

In specific examples, muscle stem cells are expanded cells in the presence of both FGF-2 and IGF-1 and in the presence of or absence of the exemplary catalytic antioxidant FBC-007 to determine the effects of catalytic antioxidants on muscle stem cell aging. The parameters shown in Table 1 are measured.

TABLE 1
Experimental Design

| skMSC Expansion | | |
|---|---|---|
| Conditions | In Vitro Aging Outcome Measures | |
| Nonsorted Populations | ROS levels (by CM- | Growth kinetics: |
| −GF expansion | H2DCFDA), | total number of |
| (baseline) | oxidative DNA damage | population doublings |
| +GF expansion, − | (8-OHdG), | (PDs), population |
| antioxidant | SOD (chromogen, | doubling time (PDT), |
| +GF expansion, + | 490 nm) GSH levels | cell division time |
| antioxidant | (monochlorobimane), | (CDT), |
| FACS-sorted | telomere length (Flow- | mitotic fraction or |
| Populations | FISH) & telomerase | % of nondividing/ |
| CMFDA+ nondividing | activity (per) | quiescent cells |
| quiescent cells | | |
| CMFDA− dividing | | |
| fraction | | |

It is determined that the addition of the antioxidant protects the human muscle stem cells from age-related changes. As a control dithiothreitol (DTT), an inhibitor of FBC-007 catalytic activity, is used to confirm that the reduction in cell damage is due to the actions of the catalytic antioxidant.

To determine if the aging of the stem cell population is related to a loss of the quiescent subpopulation with the GF-stimulated population, the level of quiescence is assessed in non-expanded populations and GF-expanded populations using time-lapsed live cell imaging.

Cells are sorted by fluorescent activated cell sorting (FACS) to separate cells based on carboxyfluorescein diacetate succinimidyl ester (CFSE) labeling into non-dividing 5-chloromethylfluorescein diacetate positive (CFMDA+) and dividing cells (CFMDA−) and confirm quiescence by live cell imaging. It is determined if the nondividing quiescent fraction represents younger cells with longer telomeres and telomerase activity. It is also determined if baseline differences exist between these subsets in terms of ROS stress and antioxidant capacity.

To determine if the quiescent/nondividing subset shows greater longevity upon activation, FACS-separated populations of cells are expanded in vitro to determine if the quiescent cells can be activated and subsequently will be capable of longer in vitro expansion as compared to expansion of cells depleted of the quiescent subset.

### Methods

Cell Isolation: Human muscle cells are isolated using methods standard in the art (NDRI tissue) (see e.g. Qu-Petersen et al., J. Cell Biol. 157, 851-64 (2002); Lee et al., J. Cell Biol. 150, 1085-100 (2000); Rando & Blau, J. Cell Biol. 125, 1275-87 (1994); Blau & Webster, Proc. Natl. Acad. Sci. U.S.A. 78, 5623-7 (1981); and Webster et al., Exp. Cell Res. 174, 252-65 (1988)).

Cytokine Stimulation: Long-term stimulation/expansion assays on human skMSCs is performed as previously described (Deasy et al., Mol. Biol. Cell 16, 3323-33 (2005), which is encorparated by reference herein in its entirety). Briefly, human skMSC populations are grown in continuous culture in EGM2 with and without medium containing human recombinant FGF-2 (0, 50, 100 ng/mL) or IGF-1 (0, 50, 100 ng/mL). Passaging is performed every 3-4 days; flow cytometry and PCR (see below) and live cell imaging (LCI) (see e.g. Schmidt et al., Industrial Robot 35, 116-124 (2008)) is performed weekly until senescence or for 16 weeks. Growth kinetics as listed in Table 1 is performed as described previously (Deasy, et al., Mol. Biol. Cell 16, 3323-33 (2005)).

Antioxidant Supplement Culture: Cultures are supplemented with 34 μM or 68 μM FBC-007. To inhibit FBC-007, to confirm action and reversibility of the antioxidant activity, DTT is used (see e.g. Bottino et al., Diabetes 53, 2559-68 (2004) and Tse et al., Free Radic. Biol. Med. 36, 233-47 (2004)).

Flow Cytometry and mRNA Analysis by PCR: The expression of and mRNA for CD45, CD34, CD56, CD 144, CD146, CD44, CD90, and CD105 is examined as previously described (Schugar et al., J. Biomed. Biotechnol. 2009, 789526 (2009)).

Karyotyping: Chromosomal karyotyping is performed as previously described (Deasy et al., Mol. Biol. Cell 16, 3323-33 (2005) and Deasy et al., J. Cell Biol. 177, 73-86 (2007)).

Telomerase activity and telomere length: As previously described (Deasy et al., Mol. Biol. Cell 16, 3323-33 (2005) and Deasy et al., J. Cell Biol. 177, 73-86 (2007)), telomerase activity is determined using the TeloTAGGG Telomerase PCR ELISA PLUS kit (Roche) according to manufacturer's protocol. For the flow cytometry-based measurement of telomere length, telomeres in the groups is detected with the PNA Kit for Flow Cytometry (DakoCytomation) according to the manufacturer's protocol.

Intracellular ROS detection by CM-H2DCFDA: The skMSCs groups listed in Table 1 are examined for their level of ROS. Cells are harvested and loaded with 5 μM FITC-conjugated 5-(and -6)-carboxy-2′,7′-dichlorodihydrofluorescein diacetate (CM-H2DCFDA dye, Moleculat Probes). Results are presented as change in percentage CM-H2DCFDA-positive cells with respect to unstimulated controls, as previously described (Urish et al., Mol Biol Cell 20, 509-20 (2009) and Sklavos et al., Free Radic. Biol. Med. 45, 1477-86 (2008)).

Superoxide Dismutase Detection: Total activity of superoxide dismutase (SOD) is measured using a colorimetric assay (Chemicon, APT290). skMSCs are homogenized using a lysis buffer (10 mM Tris, pH 7.5, 150mN NaC1, 0.1 mM EDTA, and 0.5% Triton X-100) and centrifuged at 12,000×g for 10 min to collect cell lysate. 10 μL xanthine oxidase (1:10) is added to lysates for 1-2 hrs at 37° C. Optical density is read at 490 nm.

Glutathione Level Detection: Glutathione levels are assessed as an indicator of oxidative stress using monochlorobimane (MCB) and flow cytometry as previously described (Deasy et al., J. Cell Biol. 177, 73-86 (2007)), or by use of the GSH-Glo Glutathione Assay (Promega); both methods are quantitative. In the MCB assay, cells are incubated in 5 μM monochlorobimane (Invitrogen) in normal growth media for 20 min at 37° C. and analyzed at 461 nm.

CFMDA or CFSE labeling and FACS-sorting: Cells are labeled with CFMDA/CFSE (Molecular Probes, 02925) by washing skMSCs in HBSS, centrifuging 2000 RPM for 5 minutes, then washing in PBS to remove free protein. Following centrifugation at 2000 RPM for 5 minutes, cells are re-suspended in 0.5-8.0 μM CFSE in pH=7.0 0.1% g/mL BSA in PBS. Cells are incubated at 37° C. for 10 minutes, then quenched with ice-cold media. skMSCs are then FACS-sorted to remove any unlabeled cells (see FIG. 7).

In Vitro Live Cell Imaging (LCI) to study Quiescent Nondividing cells. In vitro LCI is used to measure the fraction or proportion which is quiescent and nondividing after cytokine stimulation. The LCI system is unique in providing time-lapse images to characterize the cell populations. Using CytoTracker, DataCollector, and informatics tools, the growth rates of the populations are determined as described previously (Deasy et al., Stem Cells 20, 50-60 (2002)). In this way subpopulations or individual cells can be quantified. The combined the cell growth counts and the data of cell division time is used to calculate the size of the non-dividing fraction via a non-exponential Sherley equation (Sherley et al., Cell Prolif. 28, 137-44 (1995)).

Statistical Analysis. ANOVA analysis (parametric or non-parametric as appropriate) is performed to determine significant differences in the outcome measures (Table 1) for skMSCs which are GF-expanded with and without the catalytic antioxidant as compared to those which have not received growth factors (SigmaStat and SPSS).

### Example 3

### In Vitro Expansion Induces Stem Cell Myogenic Differentiation, which is Reduced by Anti-Oxidant Treatment

This example demonstrates that in vitro expansion lead to a loss in stem cell myogenic differentiation and that treatment with anti-oxidants protection stem cell phenotype and maintenance of myogenic activity. While this example references muscle stem cells, it is equally applicable to other stems cell types that undergo differentiation.

Several phenotype changes could be involved with GF-induced cell culture aging of human muscle cells. For example, recent reports showed that a decline in myf5 or myoD is associated with aged muscles, and this is associated with fibrosis in the tissue. If in vitro expansion has similarities to in vivo aging, then similar changes in the in vitro aged cell should also be observed.

To determine that myogenic differentiation capacity is reduced in culture expanded populations differentiation is induced in the groups listed in Table 2 and the cells in these groups are assayed for myogenic markers and differentiation using PCR and immunocytochemistry analysis.

TABLE 2
Study Design

| skMSC Expansion | | |
|---|---|---|
| Conditions | | |
| (expansions performed in | | |
| Example 2) | Functional Change Outcome Measures | |
| Nonsorted Populations | MyoD, myf5 | p53, p21, and p27, |
| −GF expansion (baseline) | and myogenin, | loss of contain |
| +GF expansion, − | desmin, myosin | inhibition, |
| antioxidant | heavy chain and | chromosome |
| +GF expansion, + | dystrophin (in vitro) | analysis (PCR, |
| antioxidant | Vimentin, collagens, | western and |
| FACS-sorted Populations | (PCR, flow and | Flow-FISH, |
| CMFDA+ nondividing | immunostainings) | giemsa staining, |
| quiescent cells | | soft agar growth) |
| CMFDA− dividing fraction | | |

It is anticipated that cells that show signs of in vitro aging will show a reduced myogenic capacity. Subsequently it is determined if a catalytic antioxidant, such as FBC-007, can alleviate this loss of function. DTT, an inhibitor of FBC-007, is used to examine reversibility and confirm that the regain in function is due to the actions of the antioxidant.

The fibrogenic phenotype of the groups listed in Table 2 are also examined and the cells are assayed for fibrogenic markers vimentin and collagen secretion using PCR and immunocyto-chemistry analysis. It is anticipated that if in vitro expansion mimics some aspects of in vivo aging, then an increase in myogenic-to-fibrogenic conversion may occur. Subsequently it is determined if FBC-007 can alleviate this loss of function. An inhibitor of FBC-007 is used to confirm that the regain in function is due to the actions of the antioxidant.

### Methods

In vitro Myogenic Differentiation: Cell populations from 3 different time points (<5 PDs, midpoint PDs, and maximal expansion) are evaluated. Briefly cells are plated at high density of 2000 cells/cm² in a low serum medium. After 3-4 days in culture, the media is changed to low serum or 2% serum medium. After day 7, immunocytochemical staining is performed to reveal fast myosin heavy chain (MyHC) expression. Methanol-fixed cultures are blocked with 5% HS and incubated with monoclonal mouse anti-MyHC (Sigma, 1:250, Sigma), biotinylated IgG (1:250, Vector), and streptavidin-Cy3 (1:500). Immunostaining also reveals MyoD, myf5 and myogenin expression. In vitro differentiation efficiency is calculated as the ratio of myogenic nuclei to total nuclei.

Soft agar assay: The ability of cells to grow in the absence of adhesion is another indicator of transformation. In an effort to evaluate the growth of human stem cells on soft agar, the cells are collected and suspended in DMEM containing 0.3% Noble agar. The suspension is then plated over a layer of solidified 0.6% Noble agar. Colony presence is assessed after 14-21 days of culturing. Colonies are counted and their size is determined. Three to 5 replications of each experiment are performed, and ANOVA is used to conduct statistical comparisons among the different passages of human cell populations.

Cell Aging: Senescence is often recognized by a shortening of the telomeres or a decrease in telomerase activity. Telomeres are 3′ single-stranded repeating DNA strings that cap chromosomes. In normal cells, telomeres shorten with successive rounds of cell division; the telomerase enzyme maintains telomere length. Senescing cells often exhibit reduced levels of the telomerase nucleoprotein complex, whereas both immortalized cells and tumorogenic cells exhibit higher levels of this complex. Flow-FISH, a method which utilizes both fluorescence in situ hybridization and flow cytometry, is used to assess telomere length and telomerase activity in human skMSCs at various levels of expansion.

Loss of cell cycle controls: Oncogenic or transformed cells often lose the ability to respond to normal cell cycle control checkpoints, such as DNA damage, reduced serum, or contact inhibition. Here UV irradiation and superconfluency is used to induce these conditions and to test the cells response to conditions which normally signal cell cycle exit or arrest. The cells' behavioral responses to these conditions are examined, and assays are used to evaluate the cells' expression of p53, p21, and p27. P53 is a senescence factor and a tumor suppressor protein. p21 and p27 are in the Cap/Kip tumor suppressor family, which regulates cell proliferation and neoplastic transformation. Healthy cells express all 3 proteins. Immunohistochemical staining or western blot analysis is completed as described previously (Li et al., Am. J. Pathol. 164, 1007-19 (2004)). Briefly, the cells are lysed, separated by 12% sodium dodecyl sulfate-polyacrylamide electrophoresis gel, and transferred to nitrocellulose membranes that will be used to perform immunostaining. Polyclonal anti-p53, -p21, or -p27 (1:100, Zymed), IgG biotinylated (1:250, Vector), streptavidin-Cy3 (1:500) is used for analysis. For western blots, proteins amounts are quantified and normalized for loading. Mouse anti-β-actin (1:8000) and protein horseradish peroxidase-conjugated secondary antibodies are used. Blots are developed using SuperSignal® West Pico Chemiluminescent substrate, and bands are visualized on X-ray film.

Chromosomal aberrations: Standard karyotyping analysis of Giemsa staining and banding techniques are used as previously described to identify any chromosomal aberrations in the expanded populations (Lee et al., J. Cell Biol. 150, 1085-100 (2000) and Deasy et al., Mol. Biol. Cell 16, 3323-33 (2005)). Metaphase preparations are made from actively dividing cells treated with colcemid. Cell populations from the different time points are examined for abnormal numbers of chromosomes and structural abnormalities.

Statistical analysis: Statistical differences are assessed by ANOVA, or appropriate nonparametric test and statistical significance is assigned at a level of P<0.05 (SPSS or SigmaStat).

### Example 4

### In Vitro Aged Muscle Stem Cells have Reduced In Vivo Muscle Regeneration Efficiency Following Transplantation of Human skMSCs to mdx/SCID Muscle

This example demonstrates that in vitro aged muscle stem cells demonstrate a reduced in vivo muscle regeneration efficiency following transplantation of human skMSCs to mdx/SCID muscle.

Ultimately, the in vivo performance of stem cells determines whether the cell population has clinical potential. Therefore, the effect of the in vitro manipulations on the in vivo performance of the cells in transplantation studies to mdx/SCID animals is determined. To answer questions regarding the role of ROS levels and ROS-generated damage cells are transplanted that have damage due to ROS damage, and similar cells that have been treated with catalytic antioxidant. To determine the role of, the level, or number of quiescent cells, FACs-separated populations of quiescent cells and actively dividing cells are transplanted. In addition the in vivo neoplastic behavior of the transplanted cells is also examined.

To determine if the in vitro expansion of skMSC reduces their ability to participate in skeletal muscle regeneration in mdx/SCID animals is due to an accumulation of age-related changes and loss of myogenic function cells are transplanted at different levels of expansion into the mdx/SCID mouse. Preliminary data suggested that muscle regeneration should be assayed by 1) total number and percentage of human marker stained nuclei in vivo, 2) percentage in satellite cell position, in fiber center, or outside sarcolemma, 3) percentage of donor cell fusion and 4) expression of human dystrophin and spectrin.

The fibrosis in transplanted muscles is also examined by assaying for vimentin and collagens both by immunostaining and using Masson's trichrome. The amount of fibrosis is assayed as previously described (Deasy et al., Mol. Ther. 17, 1788-98 (2009)). It is also determined if the treatment with catalytic antioxidants will ameliorate these age-related changes.

Subcutaneous transplantation of cells to SCID animals and assay for formation of neoplastic growth up to 120 days post transplantation is used to determine if transplantation of expanded/aged skMSC may lead to neoplastic growth in vivo using the skMSC which are expanded in Example 2.

TABLE 3
Study Design for Example 4

| | | | | |
|---|---|---|---|---|
| no | −GF | +GF | +GF | Gain-and-loss of function |
| expansion | expansion | expansion, − | expansion, + | ( for quiescence questions) |
| Control | (baseline) | antioxidant | antioxidant | CFSE [+] vs CFSE [−] |

| | |
|---|---|
| In vivo muscle | n = 6 muscles/timepoint per group |
| regeneration | t = 1, 14, 30 d |
| Myofiber | |
| contribution | |
| Fibrosis | |
| contribution | |
| In vivo neoplastic | n = 6 muscles |
| growth | t = 120 d |

Cell Transplantation and muscle repair and In Vivo Skeletal Muscle Regeneration Efficiency The stem cell populations of Table 3 are transplanted into the TA muscles of mdx/SCID mice as previously described (Qu-Petersen et al., J. Cell Biol. 157, 851-64 (2002); Deasy et al., J. Cell Biol. 177, 73-86 (2007); Jankowski et al., J. Cell Sci. 115, 4361-4374 (2002); Deasy et al., Mol. Biol. Cell 16, 3323-33 (2005); and Zheng et al., Nat. Biotechnol. 25, 1025-34 (2007). 3×10⁵ cells are transplanted to each muscle, 6 muscles per group, for human skMSCs. Specifically, 3 donors×3 doubling ages×2 timepoints×6 muscles=108 muscles or 54 animals are used. 3 doubling ages or levels of expansion are selected as cells at <5 PDs, cells at the maximal level of expansion and cells at the midpoint level of expansion. Briefly, donor cells are resuspended in 50-100 μL PBS and injected using a 30G needle. Muscle is harvested at 14 and 30 days posttransplantation. Mdx-SCID mice are used to study the muscle regeneration ability of human stem cell candidates. Mdx mice (C57BL/10ScSn-Dmd^{mdx}) and severe combined immunodeficiency mice (C57BL/6J-Prkdc^{scid}/SzJ) are obtained from Jackson Laboratory (Deasy et al., J Cell Biol 177, 73-86 (2007); Zheng et al., Nat Biotechnol 25, 1025-34 (2007); and Payne et al., Gene Ther (2005)). For immunohistochemical analysis, cross-reacting anti-mouse or anti-human dystrophin (Novocastra, DYS2/3, 1:50), biotinylated goat anti-mouse secondary Ab (Vector, 1:500) and streptavidin-Cy3 (Sigma, 1:500) is used.

In vivo Analysis and Quantification: The Regeneration efficiency of human skMSCs is determined on the basis of several endpoints: 1) total number and percentage of human chromosome or centromeric stained nuclei; 2) location and percentage in satellite cell position, in fiber center, or outside sarcolemma; 3) percentage of donor cell fusion; and 4) expression of human dystrophin and spectrin. The size of these fibers and area of regenerated muscle is measured using Northern eclipse or Image ProPlus, as previously described (Qu-Petersen et al., J Cell Biol 157, 851-64 (2002); Deasy et al., J Cell Biol 177, 73-86 (2007); Jankowski et al., J Cell Sci 115, 4361-4374 (2002); Deasy et al., Mol Biol Cell 16, 3323-33 (2005); and Zheng et al., Nat Biotechnol 25, 1025-34 (2007)). Immunohistochemical analysis for dystrophin is performed to identify the amount of skeletal muscle repair. Mouse anti-human dystrophin (Novocastra, DYS3, 1:20), biotinylated goat anti-mouse secondary Ab (Vector, 1:500) and streptavidin-Cy3 (Sigma, 1:500) is used. Engraftment efficiency is monitored by determining the overall number of dystrophin-positive fibers, their maximal diameter, the total area of the graft and the percentage of fibers which contain donor nuclei or centrally-located nuclei. Most often the regeneration index (RI, the number of new dystrophin positive myofibers per 3×10⁵ donor cells) is reported in mouse studies.

Donor Cell Fusion: After human dystrophin or spectrin staining, the positive fibers and engraftment region is examined for the presence of human and mouse specific Y-chromosome (for host). DOP-PCR labeled Y probes and pancentromeric chromosome probes are used to determine nuclei donor or species (ID Labs, CA.) as previously described (Lee et al., J Bone Joint Surg Am 83-A, 1032-9 (2001)) (Alternatively, GFP labeled cells are used). The frequency of donor cells and their fusion with host fibers is quantified at set timepoints. The engraftment efficiency of unstimulated controls versus GF-stimulated transplantations is measured and statistically compared.

In vivo neoplastic growth: Subcutaneous injections into SCID mice are performed, as done previously (Deasy et al., Mol Biol Cell 16, 3323-33 (2005)) with mMDSC, to evaluate the possible transformation of the highly expanded cells. Expanded populations of human stem cells expanded to various PD levels (<5 PDs, midpoint and maximal expansion) are transplanted subcutaneously into the lower abdomens of C57BL/6J-Prkdc SCID mice (0.3−0.4×10⁶ cells per site, n=6). Various doses of expanded cells are transplanted both subcutaneously and intramuscularly, and growth will be assessed at several time points. Tumor growth is evaluated by palpation and radiography. Mice are sacrificed 120 days after cell injection or at signs of ulceration (if observed). Sacrificed mice are dissected and evaluated for growths at the site of injection and gross enlargement of spleen and lymph tissues. The spleen, lung, and kidneys are harvested from mice that develop neoplastic growths.

### Example 5

### Control of Stem Cell Differentiation Using Catalytic Antioxidants

This example describes the determination of the effect of catalytic antioxidants on stem cell differentiation.

Umbilical cord mesenchymal stem cells (UC-MSCs) were isolated and cultured. Cells were plated in the presence and absence of catalytic antioxidant. Live cell time-lapsed imaging was used to examine proliferation and apoptosis; images were analyzed using custom software obtained from Kairos Instruments. Osteogenic differentiation was stimulated using previously described methods; Alkaline Phosphatase (ALP) staining was used to determine stem cell differentiation (see Schugar et al., J Biomed Biotechnol 2009, 789526 (2009)). Cell phenotype was examined by flow cytometry (Schugar et al., J Biomed Biotechnol 2009, 789526 (2009)). Levels of intracellular reactive oxygen species were examined using dihydroxyrhodamine 123.

Treatment of the human UC-MSCs with catalytic antioxidants did not affect the cell phenotype or expression of mesenchymal stem cell (MSC) markers CD44, CD73, CD105 or CD90. Under osteogenic conditions an increase in intracellular reactive species levels (ROS) was observed; however, the addition of the catalytic antioxidant led to significantly reduced ROS. Importantly, the UC-MSCs grown in the presence of the drug have significantly reduced levels of differentiation as compared to cells grown in the absence of the drug, demonstrating the efficacy of catalytic antioxidants as inhibitors of stem cell differentiation.

The addition of the SOD mimic to human stem cell cultures retains the stem cell phenotype by delaying stem cell differentiation. In terms of biomanufacturing, the reduction of the amount of cell differentiation prevents contamination by differentiated cells, which in turn may release signals to induce additional differentiation. Controlling this differentiation allows for larger ex vivo expansion yields through self-renewal of the cells. In addition, in terms of in vivo application, control of the timing of differentiation through small molecule drugs is critical for therapeutic function of the cells.

### Example 6

### Isolation of Cancer Stem Cells

This example describes exemplary procedures for the isolation of cancer stem cells.

Cells isolated from a tumor are assessed for CD44 and CD24 expression, which can be used to identify and isolate cancer stem cells. It has previously been observed that CD44hi/CD24lo cells comprised about 1% of a tumor population. CD44hi/CD24lo cells (e.g. cancer stem cells) are sorted into 96 well plates and expanded as single cell clones. Plates are examined daily. After expanding clonal populations, cell surface expression of CD44 and CD24 is evaluated. Typically, cancer stem cells clones rapidly reverted to a phenotype closely resembling that of an unsorted population. That is the cells become differentiated. Therefore, initially pure cancer stem cells rapidly generate a more differentiated population comprised primarily of transient amplifying cells (TAC) or TAC-like cells. A commonly used maker for cancer stem cells, as well as for embryonal stem cells, is Oct3/4 a member of the POU family of transcription factors. In some examples, to identify cancer stem cells whose fates could be easily followed, unfractionated cancer cells are stably transfected with a plasmid encoding green fluorescent protein (GFP) under the control of a 4 kb segment of the human Oct3/4 proximal promoter. It is then determined if there is a concordance between GFP expression and the CD44hi/CD24lo cancer stem cell phenotype. In some examples, cell sorting is used to isolate GFP+ cells and then these cells are evaluated for CD44 and CD24 expression. In other examples, CD44hi/CD24lo cancer stem cells are isolated and then assessed for expression of GFP. In other examples, the morphologies and sizes of cancer stem cells is compared to non-stem cells. Typically CD44hi/CD24lo cells are distinctly smaller and rounder than the bulk of the unsorted cancer cells.

### Example 7

### Treatment of Cancer Stem Cells with Catalytic Antioxidants Freezes them in a Cancer Stem Cells-Like State

As discussed above, in culture cancer stem cells typically lose their stem cell character including their CD44hi/CD24lo phenotype as the cells differentiated into TACs. However, using the methods disclosed herein it is found that the cell surface phenotype and thus the stem cell character of the cancer stem cells can be maintained, for example after >20 weeks of in vitro passage.

In some examples the stem cells are contacted with an effective amount of a catalytic antioxidant that is between about 1 μM and about 500 μM, such as about 1 μM, about 2 μM, about 4 μM, about 6 μM, about 8 μM, about 12 μM, about 16 μM, about 18 μM, about 22 μM, about 26 μM, about 30 μM, about 34 μM, about 40 μM, about 50 μM, about 60 μM, about 70 μM, about 80 μM, about 90 μM, about 100 μM, about 150 μM, about 200 μM, about 250 μM, about 300 μM, about 350 μM, about 400 μM, or about 500 μM, such as between about 1 μM and about 10 μM, between about 5 μM and about 20 μM, between about 10 μM and about 40 μM, between about 30 μM and about 50 μM, between about 40 μM and about 100 μM, between about 50 μM and about 120 μM, between about 75 μM and about 200 μM, between about 100 μM and about 250 μM, between about 200 μM and about 400 μM, or between about 350 μM and about 500 μM.

The persistence of the CD44hi/CD24lo population suggests that these cells are cancer stem cells that are “locked” or “frozen” in a cancer stem cell-like state. To determine if the CD44hi/CD24lo cells that have been locked in the undifferentiated state are cancer stem cells, de novo tumor initiation is tested. In some examples, 10⁴ of these cells are inoculated subcutaneously in nude mice; concurrently 5×10⁶ unsorted and untreated cancer cells are inoculated in a different group of animals. Increase in de novo tumor initiation from the treated cells relative to the untreated cells indicates that the cells treated by the disclosed methods are cancer stem cells that are locked in an undifferentiated state in culture.

### Example 8

### Identification of Anti-Cancer Agents Using Cancer Stem Cells Treated with Catalytic Antioxidants

It is believed that cancer stem cells are more resistant to chemotherapeutic agents than bulk cancer cells, thus at least partly accounting for the propensity of many tumors to relapse after an initial response. Thus, cancer stem cells that are locked in a cancer stem cell state using the methods disclosed herein are ideal reagents for the testing of potential chemotherapeutic agents that target cancer stem cells (for example as described in Example 7 above).

According to the teachings herein, one or more agents for the use for inhibiting cancer stem cells, for example cancer stem cell viability can be identified by contacting a cancer stem cell that has been locked in a cancer stem cell state using the methods disclosed herein with one or more test agents under conditions sufficient for the one or more test agents to alter at least one of proliferation and/or differentiation and/or viability of the cancer stem cell indicates that the test agent is an anti-cancer agent that is effective against cancer stem cells.

In some examples, a library of chemical compounds is obtained and screened for their effect on a cancer stem cell that has been locked in a cancer stem cell state using the methods disclosed herein. An agent that results in at least a 20% decrease in one or one of proliferation and/or differentiation and/or viability indicates that the test agent is an anti-cancer agent that is effective against cancer stem cells (e.g. at least 50%, at least 75%, or at least 90% decrease) will be identified as an anti-cancer agent that is effective against cancer stem cells. The identified compounds will also be used as lead compounds to identify other agents having even greater inhibitory effects cancer stem cells. For example, chemical analogs of identified chemical entities, or variant, fragments of fusions of peptide agents, are tested for their activity methods described herein. Candidate agents also can be tested in cell lines and animal models to determine their therapeutic value. The agents also can be tested for safety in animals, and then used for clinical trials in animals or humans.

In view of the many possible embodiments to which the principles of the disclosure may be applied, it should be recognized that the illustrated embodiments are only preferred examples and should not be taken as limiting the scope of the invention. Rather, the scope of the invention is defined by the following claims. We therefore claim as our invention all that comes within the scope and spirit of these claims.

We claim:

1. A method of preventing or inhibiting differentiation in vitro of a pluripotent, a multipotent or a totipotent stem cell, comprising:
contacting the pluripotent, multipotent or totipotent stem cell in vitro with an effective amount of a catalytic antioxidant, wherein the catalytic antioxidant is a porphyrin or a tetrapyrrole, or pharmaceutically acceptable salt thereof, and wherein the catalytic antioxidant is capable of preventing or inhibiting the differentiation of the pluripotent, multipotent or totipotent stem cell in vitro, thereby preventing or inhibiting the differentiation of the pluripotent, multipotent or totipotent stem cell in vitro.

2. The method of claim 1, wherein the catalytic antioxidant is FBC-007.

3. The method of claim 1, wherein the porphyrin or the tetrapyrrole is bound to a metal ion selected from the group consisting of: a manganese ion, an iron ion, a copper ion, a cobalt ion or a nickel ion.

4. The method of claim 3 wherein the metal ion is a manganese ion.

5. The method of claim 1, wherein the catalytic antioxidant is manganese substituted FBC-007.

6. The method of claim 1, wherein the stem cell is a pluripotent stem cell.

7. The method of claim 6, wherein the stem cell is a multipotent stem cell.

8. The method of claim 1, wherein the stem cell is a muscle stem cell.

9. The method of claim 1, wherein the stem cell is a mesenchymal stem cell.

10. The method of claim 9, wherein the mesenchymal stem cell is an umbilical cord mesenchymal stem cell.

11. The method of claim 1 wherein the stem cell is a cancer stem cell.

12. The method of claim 1, wherein the stem cell is a human stem cell.

13. The method of claim 1, further comprising transplanting the stem cell into a subject.

14. The method of claim 1, wherein the catalytic antioxidant is a porphyrin.

15. The method of claim 1, wherein the catalytic antioxidant is a tetrapyrrole.

16. A method for in vitro expansion of pluripotent, multipotent or totipotent stem cells while preventing or inhibiting differentiation of the pluripotent, multipotent or totipotent stem cells, comprising:
contacting the pluripotent, multipotent or totipotent stem cells with an effective amount of a catalytic antioxidant, wherein the catalytic antioxidant is a porphyrin or a tetrapyrrole, or pharmaceutically acceptable salt thereof capable of preventing or inhibiting the differentiation of the pluripotent, multipotent or totipotent stem cells; and
expanding the stem cells in expansion media that promotes the expansion of the stem cells, thereby producing an expanded population of undifferentiated stem cells.

17. The method of claim 16, wherein the expansion media is comprises one or more of epidermal growth factor (EGF), fibroblast growth factor 2 (FGF-2), insulin-like growth factor 1 (IGF-1), FLT-3 ligand, or stem cell factor (SCF).

18. The method of claim 17, wherein the catalytic antioxidant is FBC-007.

19. The method of claim 16, wherein the porphyrin or the tetrapyrrole is bound to a metal ion selected from the group consisting of a manganese ion, an iron ion, a copper ion, a cobalt ion or a nickel ion.

20. The method of claim 19, wherein the metal ion is a manganese ion.

21. The method of claim 16, wherein the catalytic antioxidant is manganese substituted FBC-007.

22. The method of claim 16, wherein the stem cells are pluripotent stem cells.

23. The method of claim 22, wherein the stem cells are multipotent stem cells.

24. The method of claim 16, wherein the stem cells are muscle stem cells.

25. The method of claim 16, wherein the stem cells are mesenchymal stem cells.

26. The method of claim 25, wherein the mesenchymal stem cells are umbilical cord mesenchymal stem cells.

27. The method of claim 16, wherein the stem cells are human stem cells.

28. The method of claim 16, wherein the stem cells are cancer stem cells.

29. The method of claims 16, further comprising transplanting the expanded population of undifferentiated stem cells into a subject.

30. The method of claim 16, further comprising:
contacting the expanded population of undifferentiated stem cells with an agent of interest; and
determining the effect of the agent of interest on expanded population of undifferentiated stem cells.

31. The method of claim 16, wherein the catalytic antioxidant is a porphyrin.

32. The method of claim 16, wherein the catalytic antioxidant is a tetrapyrrole.
